(19) United States
(12) Patent Application Publication
(10) Pub. No.: US 2022/0331479 A1
(43) Pub. Date: Oct. 20, 2022

(54) TOPICAL PAIN PATCH

(71) Applicant: Remy Biosciences, Inc., Irvine, CA (US)

(72) Inventors: Brent E. Cahill, Laguna Beach, CA (US); Mark Burleson, Irvine, CA (US); Bruce Cahill, Laguna Beach, CA (US)

(21) Appl. No.: 17/706,869

(22) Filed: Mar. 29, 2022

Related U.S. Application Data
(60) Provisional application No. 63/167,498, filed on Mar. 29, 2021.

(51) Int. Cl. A61L 15/44; A61F 13/02

(52) U.S. Cl. CPC A61L 15/44; A61F 13/0206; A61F 13/0253

(57) ABSTRACT

The disclosure provides dermal patches capable of delivering one or more pharmaceutical agents that reduce pain, where the patch can include one or more of a film, adhesive, emulsifier, tackifier, and hydrogel.

### FIELD OF THE DISCLOSURE

The disclosure relates to sublingual patches, tablets, capsules, and pills, as well as to buccal patches and dermal patches, each of which can contain a formulation providing a pharmaceutical agent such as a drug or a nutraceutical. The drug can be one or more cannabinoids.

### BACKGROUND OF THE DISCLOSURE

Dermal patches can take the form of a monolithic-style patch or a reservoir-style patch (see, US2017/0071870 of Weimann, which is incorporated herein in its entirety). Monolithic-style patch can take the form of a sandwich, where the face that is exposed to the atmosphere is a backing, where the opposite face is a release liner, and where the filling of the sandwich is a matrix that includes an adhesive and a pharmaceutical agent such as a drug or nutraceutical. Prior to applying the patch to the skin, a release liner is removed and discarded.

Regarding reservoir-style patch, the reservoir can contain a pharmaceutical agent that is a drug or a nutraceutical. The reservoir also contains a liquid carrier and a gelling agent. The reservoir can be defined by a backing and by a permeable membrane, which together assume a “ravioli” conformation. The permeable membrane is optionally coated with an adhesive that mediates binding of the adhesive to the skin. On one side of the adhesive is the permeable membrane, and on the other side is a release linter. Prior to applying the patch to the skin, a release liner is removed and discarded.

To provide some examples of dermal patches, dermal patches are used to deliver rotigotine for treating Parkinson's disease, and where the patch provides continuous drug delivery over 24 hours, resulting in plasma pharmacokinetics similar to that with continuous i.v. infusions. Rotigotine acts on dopamine receptors (see, Elshoff et al (2015) Drugs. 75:487-501). To give another example, dermal patches can provide estrogen for therapy to post-menopausal women, and to provide ethinyl estradiol and norelgestromin for contraception. The contraceptive patch is used for 7 days, and it provides systemic concentrations similar to that with a daily oral contraceptive (see, Jung et al (2013) Drugs. 13:223-233).

### SUMMARY OF THE DISCLOSURE

In embodiments, the present disclosure provides dermal patch that is capable of adhering to the skin of a human user, and also capable of delivering pharmaceutically effective amounts of each of lidocaine, capsaicin, menthol, and at least one cannabinoid to said skin, wherein said dermal patch comprises: (i) A composition that comprises lidocaine, capsaicin, menthol, and at least one kind of cannabinoid, (ii) A backing comprising a first surface that faces said composition and a second surface that faces the atmosphere, (iii) A skin adhesive, (iv) A release liner having a first surface that faces said composition and a second surface that faces the atmosphere, wherein the release liner is capable of being peeled away thereby exposing the patch's adhesive, wherein said exposed adhesive is capable of adhering to the user's skin.

In another aspect, what is provided is the above dermal patch, wherein said at least one kind of cannabinoid comprises cannabidiol (CBD). Also, what is provided is the above dermal patch, that further comprises hemp oil, and where the hemp oil is mixed with said composition that comprises lidocaine, capsaicin, menthol, and at least one kind of cannabinoid.

Moreover, what is provided is the above dermal patch, wherein said composition further comprises one or more penetration enhancers, and wherein said one or more penetration enhancers is selected from isopropyl palmitate (IPP), DMSO, 1,2 propylene glycol, isopropyl myristate (IPM), and diethylene glycol monoethylether, dihydromyricetin, diethylene glycol monoethyl ether (Transcutol®), triacetin, dipropylene glycol, isophytol, phytol, oleic acid, a terpene, ethanol, azone (azone is 1-dodecyl azepan-2-one), oleic acid, dimethylsulfoxide (DMSO), and limonene.

Furthermore, what is provided is the above dermal patch, wherein said skin adhesive takes the form of a mixture of skin adhesive and one or more penetration enhancers selected from isopropyl palmitate (IPP), DMSO, 1,2 propylene glycol, isopropyl myristate (IPM), and diethylene glycol monoethylether, dihydromyricetin, diethylene glycol monoethyl ether (Transcutol®), triacetin, dipropylene glycol, isophytol, phytol, oleic acid, a terpene, ethanol, azone (azone is 1-dodecyl azepan-2-one), oleic acid, dimethylsulfoxide (DMSO), and limonene.

In yet another embodiment, what is provided is the above dermal patch, wherein the skin adhesive comprises at least one of: (i) An acrylate pressure sensitive adhesive, (ii) A polyisobutylene (PIB) pressure sensitive adhesive, (iii) An amine-compatible silicone pressure sensitive adhesive, and (iv) An amine-compatible silicone skin adhesive comprising a trimethylsiloxy end-capped reaction product of a silanol end-blocked polydimethylsiloxane and a silicate resin.

The present disclosure also provides the above dermal patch, wherein said skin adhesive is provided as an organic solvent solution comprising from about 50 percent to about 70 percent by weight of solid adhesive in an organic solvent like heptane or ethyl acetate and having a viscosity at 20 degrees C. of from about 400 mPa-s (millipascal-seconds) to about 1300 mPa-s, from about 450 mPa-s to about 1250 mPa-s, or from about 500 mPa-s to about 1200 mPa-s.

In another aspect, the disclosure provides the above dermal patch that is a monolithic-style dermal patch. Also provided is the above dermal patch that is a monolithic-style dermal patch comprising a matrix formulated to maintain adhesion of the dermal patch to the user's skin for a period of at least 24 hours, wherein the release liner of said monolithic-style dermal patch is releasably adhered to the matrix.

In yet another embodiment, provided is the above dermal patch that is a reservoir-style dermal patch. Also provided is the above dermal patch that is a reservoir-style dermal patch that comprises a hydrophilic porous membrane, wherein the backing and the hydrophilic porous membrane are attached to one another to define a closed volume that acts as a reservoir, wherein said composition is disposed in the reservoir, wherein said hydrophilic porous membrane has a first side that contacts said reservoir, and wherein the hydrophilic porous membrane has a second side that faces away from the backing and is coated with skin adhesive,

Moreover, the present disclosure provides the above dermal patch wherein the release liner comprises an occlusive polymeric film, such as polyester, polypropylene. In another aspect, what is provided is the above dermal patch wherein the release liner is coated with a release coating that is releasably adherable to acrylate, polyisobutylene, and silicone adhesives.

### BRIEF DESCRIPTIONS OF THE DRAWINGS

FIG. 1 is a reservoir-style dermal patch.

FIG. 2 is a monolithic-style dermal patch.

FIG. 3 shows efficacy of the embodiments of the present invention.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference.

Glossary. The words “preparation,” “composition,” and “formulation,” are used herein to refer to the same thing, unless dictated otherwise explicitly or by the context. These words refer, for example, to a liquid, a slurry, or a paste, that contains one or more pharmaceutically active ingredients. The term, “applicant's patch,” or “the patch” refers to a patch made by REMY Biosciences. The term “Terocin” refers to a patch manufactured by Alexo, Inc., Los Angeles, Calif. “Cold flow” is a creep phenomenon that occurs at relatively low temperatures (for example, room temperature) in which the drug-in-adhesive polymer oozes outside the area of contact between the epidermis and the patch. The main problem in this occurrence is that the adhesive polymer is exposed to the atmosphere, and small amounts of dust and fibers can collect on the adhesive, resulting in an unsightly black ring. Cold flow also results in slightly poorer adhesion of the patch at the edges, although this was less of a problem. See, Miyazaki, Kanno, Izutsu (2020) Cold Flow Evaluation in Transdermal Drug Delivery Systems by Measuring the Width of the Oozed Adhesive. AAPS PharmSciTech 21:120.

The term “form factor” refers to the size of the patch. A more technically correct description is, “Each adhesive patch product is a 50 square centimeters drug-in-adhesive transdermal patch product, intended for patient use over a 24-hour cycle.”

### DETAILED DESCRIPTIONS OF THE DRAWINGS

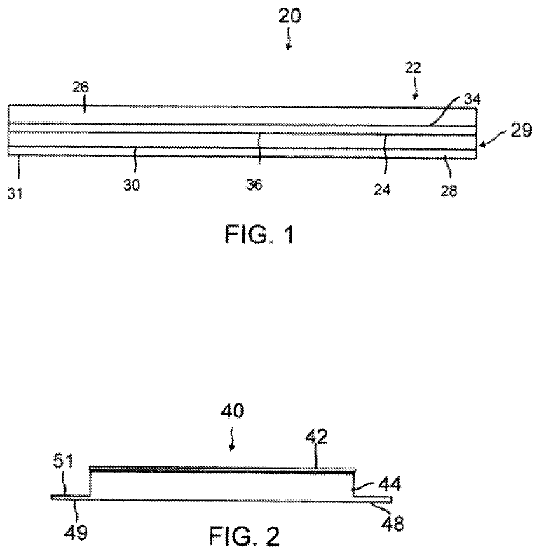

Referring to FIG. 1, a reservoir-style transdermal delivery device 20 for the transdermal delivery of CBD is depicted. Reservoir-style transdermal delivery device 20 comprises a backing 22 and a hydrophilic, porous membrane 24. The backing 22 and hydrophilic, porous membrane 24 are attached to one another so as to define a closed volume which acts as a reservoir 26. A preparation comprising CBD, a liquid carrier, and a gelling agent is disposed in the reservoir 26. First side 34 of the hydrophilic, porous membrane 24 is in contact with the preparation. A second side 36 of the hydrophilic, porous membrane 24 faces away from backing 22 and is coated with a skin adhesive 30.

The skin adhesive 30 is preferably formulated to adhere the device 20 to the user's skin for a period of no less than about twenty four hours while avoiding appreciable skin irritation to the user's skin. Preferred skin adhesives 30 include polyisobutylene adhesives, acrylate adhesives, or a blend of the two. The skin adhesive is preferably provided as an organic solvent solution comprising from about 20 percent to about 70 percent by weight of solid adhesive in an organic solvent like heptane or ethyl acetate and having a viscosity at 20 degrees C. from about 400 mPa-s to about 8000 mPa-s, preferably from about 850 mPa-s to about 7000 mPa-s, and more preferably from about 1250 mPa-s to about 6500 mPa-s.

A first surface 29 of a release liner 28 is releasably adhered to skin adhesive 30, and a second surface 31 of release liner 28 faces away from skin adhesive 30. Suitable release liners include occlusive polymeric films, such as polyester, polypropylene, coated with a release coating that is releasably adherable to acrylate, polyisobutylene, and silicone adhesives. Suitable release coatings on first surface 29 of release liner 28 include fluoropolymers and silicone polymers. Commercially available, coated release liners that are suitable for use as release liner 28 include Scotchpak® 1022, 9741, 9744, 9748, and 9755 supplied by 3M of Minneapolis, Minn., and FRA 314 and 315 supplied by Fox River Co. To use the reservoir transdermal device 20, release liner 28 is peeled away from skin adhesive 30, thereby exposing skin adhesive 30, and the device 20 is applied so that the skin adhesive 30 contacts the user's skin.

Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 24 that is preferably from about 10 to about 20 g/square meter, more preferably from about 12-18 g/square meter, and still more preferably from about 14-16 g/square meter.

Hydrophilic, porous membrane 24 preferably has a mean flow pore size of no more than about 1 micron, preferably not more than about 0.8 microns, still more preferably no more than about 0.4 microns, and even more preferably no more than about 0.2 microns. At the same time, porous membrane 24 preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233, the entirety of which is hereby incorporated by reference.

In the same or other examples, hydrophilic porous membrane 24 preferably has a porosity of at least about 60 percent, more preferably at least about 65 percent, and still more preferably at least about 70 percent. At the same time, hydrophilic porous membrane 24 preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane 24 preferably has a thickness of no more than about 50 microns, preferably no more than about 40 microns, and even more preferably no more than about 35 microns. At the same time, hydrophilic porous membrane 24 preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, hydrophilic porous membrane 24 preferably has an air permeability as determined by the Gurley Test Method (according to ISO 5636-5) that is preferably at least about 10 sec/50 ml, more preferably at least about 20 sec/50 ml, and still more preferably at least about 25 sec/50 ml. At the same time, hydrophilic porous membrane 24 preferably has an air permeability of no more than about 50 sec/50 ml, more preferably no more than about 40 sec/50 ml, and still more preferably no more than about 35 sec/50 ml.

In the same or other examples, hydrophilic porous membrane 24 preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane 24 preferably has a percent elongation in the machine direction as determined by ASTM D882-12 that is preferably at least about 10 percent, more preferably at least about 15 percent, and still more preferably at least about 20 percent.

Hydrophilic porous membrane 24 preferably comprises at least one polymeric material. In one example, hydrophilic porous membrane 24 comprises a polyolefin polymer and a hydrophilic component that comprises a hydrophilic polymer and optionally, a surfactant. As used herein, the term “hydrophilic” when used to describe a porous membrane refers to a membrane that at 20 degrees C. provides a water flux for demineralized water through the membrane of at least (0.5 liters) divided by ((meters squared)(hbar)).

The content of the polyolefin polymer is preferably less than or equal to 98 percent by weight based on the total dry weight of the membrane 24, and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

One example of a commercially available hydrophilic, porous membrane that is suitable for use as hydrophilic, porous membrane 24 is supplied by Lydall Performance Materials B.V. under the name Evopor® 5E02A. Evopor® 5E02A is a porous hydrophilic membrane comprising a polyethylene support and a poly (ethyl vinyl) alcohol hydrophilic component.

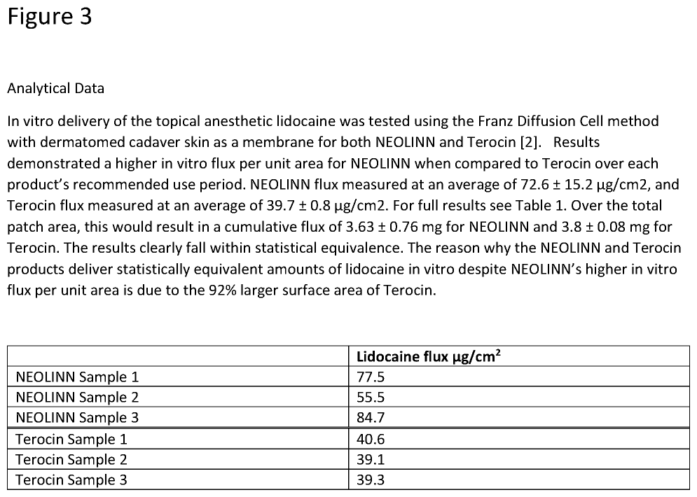

Referring now to FIG. 3, in vitro delivery of the topical anesthetic lidocaine was tested using the Franz Diffusion Cell method with dermatomed cadaver skin as a membrane for both applicant's patch and Terocin [2]. Results demonstrated a higher in vitro flux per unit area for applicant's patch when compared to Terocin over each product's recommended use period. applicant's patch flux measured at an average of 72.6±15.2 μg/cm2, and Terocin flux measured at an average of 39.7±0.8 μg/cm2. For full results see Table 1. Over the total patch area, this would result in a cumulative flux of 3.63±0.76 mg for applicant's patch and 3.8±0.08 mg for Terocin. The results clearly fall within statistical equivalence. The reason why the applicant's patch and Terocin products deliver statistically equivalent amounts of lidocaine in vitro despite the applicant's patch higher in vitro flux per unit area is due to the 92% larger surface area of Terocin.

As stated previously, preparation comprises CBD and a liquid carrier. In certain examples, the polar organic liquid comprises a molecule having one or more carboxylic acid groups. In the same or other examples, the polar organic liquid comprises a molecule having one or more hydroxyl groups. Suitable polar organic liquids comprising one or more hydroxyl groups include those comprising between 2 and 30 carbon atoms per molecule, including without limitation, ethanol. Suitable polar organic liquids comprising one or more carboxylic acid groups include fatty acids, including without limitation oleic acid. Liquid carriers comprising ethanol and/or oleic acid are preferred, and liquid carriers comprising oleic acid are especially preferred. Suitable liquid carriers also include mixtures of polar organic liquids and water. Examples of such mixtures include mixtures of ethanol and water. In ethanol/water mixtures, the maximum concentration of water is preferably about ten (10) percent by weight of the total amount of ethanol and water.

Preparation also may comprise of a gelling agent which makes the preparation thixotropic. Suitable gelling agents include: sodium carboxymethyl cellulose, hydroxypropyl cellulose, hydroxyethyl cellulose, polyacrylic acid, methyl cellulose, xanthan gum, and so on. In certain examples, cellulose gelling agents such as hydroxyethylcellulose are preferred. The gelling agent increases the viscosity of and provides structural integrity to preparation, which improves the ease of placing and retaining preparation in reservoir 26 before the reservoir 26 is closed by heat sealing the hydrophilic, porous membrane 24 to the occlusive backing 22. The gelling agent is preferably pharmacologically inactive.

The CBD is present in a therapeutically effective amount in preparation. A “therapeutically effective amount” is an amount of CBD sufficient to achieve a desired therapeutic effect over a desired time period. The CBD may be provided as substantially pure CBD, such as in a powder form, or as part of an oil extract comprising other cannabinoids. Presently (2015) marijuana growers extract CBD from plants in form of oils or “shatters” that have content of CBD ranging fern 25 to 80% by weight of the oil and THC from 10-25% by weight of the oil. Recently, however, due to availability of sophisticated separators, it made possible to obtain pure CBD of 99.9% from hybrid marijuana plants or hemp plants with very low content of THC below 0.1%.

In certain examples, preparation preferably comprises from about one (1) percent to about fifty (50) percent by weight CBD, more-preferably from about five (5) to about 30 percent by weight CBD, and more preferably from about 10 to about 20 percent by weight CBD.

In the same or other examples, the preparation comprises a liquid carrier in amounts ranging from about 50 percent to about 99 percent by weight of the preparation. Preparation also comprises from about one (1) percent to about ten (10) percent, and preferably about three (3) percent of a gelling agent (preferably hydroxyl propyl cellulose) by weight of the preparation. Preparation also includes from zero to about ten (10) percent by weight of at least one penetration enhancer. In certain examples, the liquid carrier comprises at least one polar liquid of the type described previously. Suitable penetration enhancers include 1,2 propylene glycol, dimethyl sulfoxide (DMSO), oleic acid, and isopropyl palmitate (IPP).

When provided as substantially pure CBD, the amount of CBD in preparation by weight of CBD plus liquid carrier(s) (i.e., excluding gelling agents such as cellulose derivatives like hydroxyl propyl cellulose), preferably ranges from about two (2) percent to about 40 percent by weight of the combination of CBD and liquid carrier(s) more preferably from four (4) percent to about 30 percent by weight of the combination of CBD and liquid carrier(s), and still more preferably from about five (5) percent to about 20 percent by weight of the combination of CBD and liquid carrier(s). When CBD is provided as part of a Cannabis plant oil extract, the concentration of oil in the combination of oil and liquid carrier(s) is preferably from about five (5) percent to about, fifteen (15) percent by weight of the combination of oil and liquid carrier(s), more preferably from about eight (8) percent to about thirteen (13) percent by weight of the combination of oil and liquid carrier(s), and still more preferably from about nine (9) to about eleven (11) percent by weight of the combination of oil and liquid carriers(s). Ten percent oil by weight of the combination of oil and liquid carrier(s) is especially preferred.

Suitable backing materials for backing 22 include occlusive polymeric films such as polyethylene, polyethylene terephthalate (PET) and combinations thereof. Although device 20 may include an overlay patch, in preferred examples, one is not provided. In general, an overlay patch is not necessary if the hydrophilic, porous membrane 24 is already coated with skin adhesive 30. If the membrane 24 is not coated with an adhesive (e.g., in order to maximize the flux of CBD into the skin), an overlay patch should be placed over the reservoir 26 in order to ensure intimate contact of the hydrophilic, porous membrane 24 with skin. In certain examples, the skin contact area (“active transdermal flux area”) of the membrane 24 of a device 20 is preferably at least about 10 square centimeters, more preferably at least about 20 square centimeters and still more preferably at least about 30 square centimeters. At the same time, the skin contact area of device 20 is preferably no more than about 30 square cm, preferably no more than about 25 square cm, and still more preferably no more than about 22 square cm. At a given flux rate, the skin contact area may be selected to achieve the desired daily dose of CBD (or the dose over whatever time period is of therapeutic interest).

Referring to FIG. 2, an example of monolithic-style transdermal drug delivery device 40 for delivering CBD is depicted. Monolithic transdermal device 40 includes a backing 42 of the type described previously with respect to backing 22 of reservoir transdermal device 20. A matrix 44 of skin adhesive mixed with a therapeutically effective amount of CBD is coated on one side of backing 42. The matrix 44 is preferably formulated to adhere the device 20 to the user's skin for a period of no less than about 24 hours while avoiding appreciable skin irritation to the user's skin. A release liner 48 is releasable adhered to matrix 44 on a surface of matrix 44 opposite the surface adhered to backing 42. First side 49 of release liner 48 faces away from matrix 44 and a portion of second side 51 of release liner 48 is adhered to matrix 44. To use the monolithic transdermal device 40, the release liner 48 is peeled away and the exposed surface of adhesive matrix 44 is applied to the skin.

The skin adhesive comprising matrix 44 preferably comprises at least one of an acrylate pressure sensitive adhesive, a polyisobutylene pressure sensitive adhesive, and an amine-compatible silicone pressure sensitive adhesive. Suitable acrylate adhesives include DuroTak 87-2074 and DuroTak 87-2194. Suitable polyisobutylene adhesives include those having a viscosity-average molecular weight ranging from about 30,000 Daltons to about 70,000 Daltons, preferably from about 35,000 Daltons to about 65,000 Daltons, and more preferably from about 40,000 Daltons to about 60,000 Daltons. Suitable polyisobutylene adhesives also have a viscosity at 20 degrees C. ranging from about 1000 mPa-s to about 7000 mPa-s.

In certain preferred examples, matrix 44 preferably comprise a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 44, more preferably from about two (2) to about six (6) percent by weight of matrix 44, and still more preferably from about three (3) to about four (4) percent by weight of the matrix 44. Mineral oils that are suitable for use as the adhesion/viscosity modifier have a molecular weight ranging from 100 to about 1000 Daltons, more preferably from about 200 to about 600 Daltons, even more preferably from about 350 Daltons to about 450 Daltons, and still more preferably about 400 Daltons. Silicone fluids that are suitable for use as the adhesion/viscosity modifier preferably comprise —OH end-capped polydimethylsiloxanes having a kinematic viscosity at 20 degrees C. ranging from about 100 cSt to about 1000 cSt. Commercially available silicone fluids that may be used as the adhesion/viscosity modifier include the Dow Corning Q7-9120 fluids, which are available in kinematic viscosities (at 20 degrees C.) of 20, 100, 350, 1000, and 12,500 cSt. In preferred examples of silicone adhesion/viscosity modifier, the Q7-9120 100 cSt or 1000 cSt for mixtures thereof) are used.

Preferred polyisobutylene adhesives are not supplied with mineral oil. In certain preferred examples, the polyisobutylene component of matrix 44 is a Vistanex LM polyisobutylene adhesive. In other preferred examples, the polyisobutylene component of matrix 44 is an Oppanol B13 polyisobutylene adhesive supplied by BASF.

In yet another example, the adhesive component of matrix 44 may comprise a blend of acrylic adhesive and polyisobutylene adhesive, and preferably, a blend of an acrylic adhesive and a polyisobutylene adhesive having the viscosity-average molecular weight described above (from about 30,000 Daltons to about 70,000 Daltons, preferably from about 35,000 Daltons to about 65,000 Daltons, and more preferably from about 40,000 Daltons to about 60,000 Daltons). When acrylic adhesives are combined with such polyisobutylene adhesives, the amount of acrylic adhesive by weight of the total amount of adhesive in matrix 44 is preferably from about one (1) to about 50 percent. In one example, the adhesive component of matrix 44 comprises 80 percent Oppanol B13 by weight of the total amount of adhesive in matrix 44 and twenty (20) percent Durotak 87-2516 by weight of the total amount of adhesive in matrix 44.

In examples in which matrix 44 comprises an amine-compatible silicone adhesive, the amine-compatible silicone adhesive is preferably of the type described previously with respect to skin adhesive 30 of reservoir transdermal device 20.

The amount of CBD in the matrix 44 preferably ranges from about one (1) to about 30 percent by weight of the matrix 44, more preferably from about two (2) percent to about 25 percent by weight of the matrix 44, and still more preferably from about five (5) percent to about twenty (20) percent by weight of the matrix 44. The amount of Cannabis compounds other than CBD is preferably less than about one (1) percent. In those cases where pure CBD is used in matrix 44, the amount of pure CBD by weight of matrix 44 is preferably from about two (2) percent to about twenty (20) percent, more preferably from about four (4) percent to about fifteen (15) percent, and still more preferably from about five (5) percent to about ten (10) percent.

When CBD is provided as part of a Cannabis plant oil extract, the amount of oil by weight of matrix 44 is preferably from about fifteen (15) percent to about 40 percent by weight, preferably from about twenty (20) percent to about 30 percent by weight, and still more preferably from about 24 percent to about 26 percent by weight in such cases, the CBD content as a percentage of the Cannabis plant oil extract is preferably from about 25 percent to no more than about 50 percent, more preferably from about 30 percent to about 50 percent, and still more preferably from about 40 percent to about 50 percent by weight of the Cannabis oil.

In certain preferred examples wherein CBD is provided as part of a Cannabis plant oil extract, the oil further comprises rosins and/or terpenes that remain present after extraction. It has been found that these rosins and terpenes improve adhesion to the skin. Thus, the use of plant extract oils in monolithic device 40 provides a synergistic effect in both allowing for transdermal delivery of CBD and providing a device 40 that can withstand showers and minor brushings over the worn device 40 for many days.

Monolithic device 40 may also include penetration enhancers, including but not limited to diethylene glycol monoethyl ether (Transcutol®) oleic acid, isopropyl palmitate (IPP), DMSO, 1,2 propylene glycol, and isopropyl myristate (IPM). The amount of penetration enhancer preferably ranges from zero to about ten (10) percent by weight of the matrix 44.

In certain examples, the skin contact area of device 20 is preferably at least about 10 square cm, more preferably at least about 15 square cm, and still more preferably at least about 18 square cm. At the same time, the skin contact area of device 20 is preferably no more than about 30 square cm, preferably no more than about 25 square cm, and still more preferably no more than about 22 square cm. At a given flux rate, the skin contact area may be selected to achieve the desired daily dose of CBD (or the dose over whatever time period is of therapeutic interest).

Pharmaceutical Agents, Such as Cannabinoids, Lidocaine, Menthol, and Capsaicin.

The present disclosure provides compositions, formulations, and preparations, as well as dermal patches comprising said composition, formulation, or preparation, and medical devices containing said composition, formulation, or preparation. In a preferred embodiment, the lidocaine, menthol, capsaicin, and CBD are all mixed together to produce a mixture. Optionally, this mixture can also include one or more of ascorbyl palmitate, an enhancer, and a matrix. This matrix can comprise a hydrogel, an adhesive, or the combination of a hydrogel and an adhesive.

Said composition, formulation, or preparation, can comprise one or more of the following cannabinoids: cannabidiol (CBD), cannabichromene (CBC), cannabigerol (CBG), delta-9-tetrahydrocannabinol (delta-9-THC), and cannabinol (CBN), tetrahydrocannabinovarin (THCV), which is a propyl analogue of THC, and cannabidivarin (CBDV), which is a propyl analogue of CBD.

In exclusionary embodiments, the composition, formulation, or preparation, can exclude one or more of lidocaine, menthol, capsaicin, CBD, CBC, CBG, delta-9-THC, CBN, THCV, and CBDV. In other exclusionary embodiments, composition, formulation, preparation, and dermal patch that comprises said composition, formulation, or preparation, and medical device that comprises said composition, formulation, or preparation, can exclude compositions, dermal patches, and medical devices, that comprise one or more of the terpenes that are identified herein.

Regarding menthol, experiments on TRPM8 channels revealed a mechanism of action for menthol's ability to produce both a cooling sensation and reduction in pain. This mechanism involves menthol's ability to activate transient receptor potential melastatin-8 (TRPM8) channels. Menthol induces the sensation of cooling by activating TRPM8, an ion channel in cold-sensitive peripheral sensory neurons. To summarize, menthol-induced analgesia of acute and inflammatory pain is mediated by TRPM8.

Regarding lidocaine, lidocaine's anti-pain action results from mechanism of action where the drug blocks voltage-gated sodium channels (VGSCs), leading to a reversible block of action potential propagation. Intracellularly, the ionized form blocks sodium channels by binding to Segment 6 of Domain 4 of the alpha-subunit of the ion channel. When lidocaine is bound to the sodium channel, the influx of sodium ions is interrupted and inhibits the action potential generation and inhibits propagation of the action potential. Further regarding lidocaine's mechanism of action, lidocaine interacts with peripheral and central voltage-gated sodium channels, at the intracellular side of the cell membrane.

Regarding capsaicin, the mechanism of action in pain relief includes the following. Capsaicin is an agonist of TRPV1. Capsaicin activates TRPV1-expressing nociceptors on the skin, causing the onset of pain and erythema. Following this action, capsaicin reduces pain through a process called “defunctionalization” of nociceptor fibers. Defunctionalization is the cellular consequence of calcium influx triggered by capsaicin-activated TRPV1. TRPV1 means, “transient receptor potential vanilloid-1.” TRPV1 is a member of a family of Transient Receptor Potential (TRP) ion channels

In studies on human subjects, pain can be rated using the numerical pain rating scale (NPRS), using the Pain Intensity Visual Analog Scale, or using the Pain Relief Verbal Response Scale, and the Global Medication Performance Scale (see, Farrar, Young, Werth (2001) Pain 94:149-158; Farrar, Polomano (2010) Anesthesiology. 112:1464-1472). The Memorial Pain Assessment Scale and the WOMAC pain scale are additional pain scales used with human subjects (see, pages 71 and 232 of Tom Brody (2016) Clinical Trials, 2nd ed., Elsevier Press, San Diego).

The present disclosure also provides dermal patches that do not contain any composition or formulation, and also provides medical devices that do not contain any composition or formulation.

Preferred formulations include one or more cannabinoids. The major cannabinoids from Cannabis sativa are cannabidiol (CBD), cannabichromene (CBC), cannabigerol (CBG), delta-9-tetrahydrocannabinol (delta-9-THC), and cannabinol (CBN) (Appendino (2008) J. Nat. Prod. 71:1427-1430). Clinical trials have established that formulations derived from Cannabis, can improve neuropathic pain of multiple sclerosis, improve appetite and sleep quality in cancer patients, relieve pain in fibromyalgia patients, and serve as an anti-emetic for chemotherapy induced nausea and vomiting (see, Health Canada (February 2013) Information for Health Care Professionals. Cannabis (Marihuana, Marijuana) and the Cannabinoids (152 pages)). The present disclosure also provides tetrahydrocannabinovarin (THCV), which is a propyl analogue of THC, and cannabidivarin (CBDV), which is a propyl analogue of CBD.

What is provided are formulations and compositions that include both THC and CBD at a given ratio are provided, such as at the ratio of about 95/5, about 90/10, about 80/20, about 70/30, about 60/40, about 50/50, about 40/60, about 30/70, about 20/80, about 10/90, and about 5/95 (by weight). Administering formulations containing both THC and CBD can have greater influence on reducing pain that formulations containing only THC or only placebo. In exclusionary embodiments, what can be excluded is a composition, a dermal patch, a sublingual patch, a buccal patch, a capsule, or a pill, and related methods, that comprise any one or more of the above pharmaceutical agents, and can also exclude any of the above pharmaceutical agents at any of the above-recited ratios.

Hemp Oil Embodiments

In embodiments, compositions, formulations, dermal patch comprising a composition or formulation, medical device comprising a composition or formulation, and related methods, comprises hemp oil. What is provided is a mixture where hemp oil constitutes 5%, 10%, 15%, 20%, 25%, 30%, 35%, 40%, 45%, 50%, 55%, 60%, 65%, 70%, 75%, 80%, 85%, 90%, or 95%, by weight of the entire mixture (the entire mixture being the combination of the hemp oil plus said composition or formulation). This mixture can take the form of the hemp oil combined with any composition that is disclosed herein, or where hemp oil is combined with any formulation that is disclosed herein.

What is also provided is a mixture where hemp oil constitutes about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, by weight of the entire mixture (the entire mixture being the combination of the hemp oil plus said composition or formulation). In this context, the word “about” can mean plus or minus one percent, plus or minus two percent, plus or minus three percent, or plus or minus four percent.

In exclusionary embodiments, a composition, dermal patch comprising a composition, medical device comprising a composition, of the present disclosure can exclude any composition, dermal patch, or medical device that contains hemp oil at one or more of the above-recited concentrations.

Where a hydrogel or other matrix is present in said mixture, the percentage of hemp oil and the percentage of the composition in said mixture, applicant reserves the right to define the relative weights of hemp oil and the composition while ignoring the weight of the matrix. However, in some situations, it may be more convenient to define the weight of the hemp oil by comparing this weight to the total weight of the combination of the hemp oil plus matric plus the composition.

Hemp oil is available from, e.g., LyFeBaaK, Albuquerque, N. Mex., CBD American Shaman, Weymouth, Mass., and PureKana, Scottsdale, Ariz. Marijuana and hemp are two different strains of the same plant, Cannabis sativa. Phytocannabinoids such as THC and CBD, as well as terpenoids like beta-caryophyllene (BCP) and limonene, are enriched in the flowers and leaves. In contrast, the seeds of the Cannabis sativa are low in phytocannabinoids and are rich in omega-3 and omega-6 essential fatty acids, and are rich in gamma-linolenic acid, and other antioxidants. Cannabis sativa contains the psychoactive tetrahydrocannabinol (THC), and the non-psycoactive cannabidiol (CBD). These compounds are synthesized and stored in stalked glandular trichomes. Trichomes are tiny secretory epidermal glands that are present and abundant on the inflorescence of the female plant, but are present in lower numbers on leaves and stems, and absent on roots and seeds. Hemp oil can be obtained from the seeds by using a cold press. Also, hemp oil can be obtained from seeds by ultrasound extraction of hemp seeds in a solvent. Suitable solvents include, isopropanol, hexane, and a mixture of hexane:isopropanol (50:50). See, Kenari and Dehghan (2020) Food Science and Nutrition. 8:4976-4986.

Terpenes

Examples of terpenes and their classification are as follows (see, US2015-0080265): Hemiterpenes: Examples of hemiterpenes are 2-methyl-1,3-butadiene, hemialboside, and hymenoside. Monoterpenes: pinene; alpha-pinene, beta-pinene, cis-pinane, trans-pinane, cis-pinanol, trans-pinanol, limonene; linalool; myrcene; eucalyptol; alpha-phellandrene; beta-phellandrene; alpha-ocimene; beta-ocimene, cis-ocimene, ocimene, trans-ocimene, delta-3-carene; fenchol; sabinene, borneol, isoborneol, camphene, camphor, phellandrene, alpha-phellandrene, alpha-terpinene, geraniol, linalool, nerol, menthol, myrcene, terpinolene, alpha-terpinolene, beta-terpinolene, gamma-terpinolene, delta-terpinolene, alpha-terpineol, trans-2-pinanol. Sesquiterpenes: caryophyllene; beta-caryophyllene, caryophyllene oxide, humulene, alpha-humulene, alpha-bisabolene; beta-bisabolene; santalol; selinene, nerolidol, bisabolol, alpha-cedrene, beta-cedrene, beta-eudesmol, eudesm-7(11)-en-4-ol, selina-3,7(11)-diene, guaiol, valencene, alpha-guaiene, beta-guaiene, delta-guaiene, guaiene, farnesene, alpha-farnesene, beta-farnesene, elemene, alpha-elemene, beta-elemene, gamma-elemene, delta-elemene, germacrene, germacrene A, germacrene B, germacrene C, germacrene D, germacrene E. Diterpenes: oridonin. Triterpenes: ursolic acid; oleanolic acid. Additional terpenes include, alpha-cedreen, guaia-1(10),11-diene, lindestrene, and furanoeudesma-1,3-diene.

In exclusionary embodiments, compositions, formulations, dermal patch comprising said compositions or formulations, medical device comprising said compositions or formulations, of the present disclosure, and related methods of the present disclosure, can exclude any composition, formulation, dermal patch, medical device, or method that comprises one or more of the above-disclosed terpenes.

Measuring Cannabinoids

Cannabinoids can be separated, purified, analyzed, and quantified by a number of techniques. Available equipment and methods include, e.g., gas chromatography, HPLC (high pressure liquid chromatography, high performance liquid chromatography), mass spectrometry (MS), time-of-flight mass spectrometry, gas chromatography-mass spectrometry (GC-MS), and liquid chromatography-mass spectrometry (LC-MS). Equipment for separation and analysis is available from Waters Corp., Milford, Mass.; Agilent, Foster City, Calif.; Applied Biosystems, Foster City, Calif.; and Bio-Rad Corp., Hercules, Calif. Methods, equipment, and manufacturers for HPLC fractionation and identification of cannabinoids are disclosed (see, e.g., Peschel W (2016) Quality control of traditional Cannabis tinctures. Sci. Pharm. 84:567-584; Scheidweiler K B et al (2012) Simultaneous quantification of free and glucuronidated cannabinoids in human urine by liquid chromatography tandem mass spectrometry. Clin. Chim. Acta. 413:1839-1847; Singh, Guo, and Kitts (2020) Scientific Reports. 10:10567).

Biochemical properties of cannabinoids, binding to cannabinoid receptors, terpenes and terpene receptor binding, can be assessed using labeled cannabinoids, labeled terpenes, and labeled ligands where a cannabinoid or a terpene influences binding properties of the labeled ligand. Useful labels include radioactive labels, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

Cannabinoid Numbering Systems

The present disclosure uses the nomenclature as set forth by Pertwee R G et al (2010) International Union of Basic and Clinical Pharmacology. LXXIX. Cannabinoid receptors and their ligands: beyond CB1 and CB1. Pharmacol. Rev. 62:588-631. Regarding different numbering systems for the same compound, Aviv (US 2004/0110827) states that: “It should be noted that for historical reasons, these cannabinoid analogs are still named following the previous nomenclature, where the terpenic ring was the base for the numbering system. Then the chiral centers of THC type cannabinoids were at carbon atoms 3 and 4. The accepted nomenclature is now based on the phenolic ring as the starting point for numbering. Thus, THC that was previously described as delta-1-THC was later renamed delta-9-THC, similarly delta-6-THC was renamed delta-8-THC, and the chiral centers are at carbons 6a and 10a.” AVIV also has this comment about enantiomers: “delta-9-THC was established by Mechoulam R. et al. in 1967 and found to be of (-)-(3R,4R) stereochemistry. It was later found that the psychotropic activity of cannabinoids resides in the natural (3R,4R) OH series, while the opposite enantiomeric synthetic series (3S,4S) was free of these undesirable effects.”

According to Agurell (1988) Pharmacological Revs. 38:21-43, the terpene numbering system uses delta-1-THC, while the dibenzopyran system uses delta-9-THC to refer to the same chemical. Both of these numbering systems can be used for THC, CBD, and CBN.

According to Chulgin, the numbering system most broadly used recognizes both the terpene nature and the aromatic nature of the two different parts of the cannabinoid. Here, the terpene is numbered from the ringcarbon that carries that branched methyl group, and this is numbered 7, and the remaining three carbons of the isopropyl group are then numbered sequentially. The advantage to this numbering system is that this numbering system is applicable whether the center ring is closed or open. Other numbering systems are the biphenyl numbering system, the Chemical Abstracts system (substituted dibenzopyran numbering), and the Todd numbering system (pyran numbering) (see, Chulgin AT (1969) Recent developments in Cannabis chemistry. J. Psychedelic Drugs. pp. 397-415.

Matrix Embodiments

An excipient useful for granulating agents and sprays is the polyvinylpyrrolidone copolymer having a given ratio, or range of ratios, of polyvinylpyrrolidone/vinyl acetate (PVP/VA). The present disclosure provides PVP/VA (or combinations of any two polymers), at a ratio of 10/90, 20/80, 30/70, 40/60, 50/50, 60/40, 70/30, 80/20, 90/10, as well as a combination of any two polymer at a ratio of about 10/90, about 20/80, about 30/70, about 40/60, about 50/50, about 60/40, about 70/30, about 80/20, about 90/10. Also, the present disclosure can exclude PVP/VA compositions (or it can exclude a combination of any two polymers) with a ratio of, 10/90, 20/80, 30/70, 40/60, 50/50, 60/40, 70/30, 80/20, 90/10, or about 10/90, about 20/80, about 30/70, about 40/60, about 50/50, about 60/40, about 70/30, about 80/20, about 90/10, and the like. The PVP/VA copolymer has the ability to distribute homogeneously around an active ingredient during formation of an aqueous liquid phase (see, US2016/0058866 of Sekura). Polymers and copolymers are available from Sigma-Aldrich, St. Louis, Mo., Nippon Shokubai Co., Ltd., Osaka, Japan, BASF Corp., Florham Park, N.J., and Ashland, Schaffhausen, Switzerland.

In methods of manufacturing embodiments, monolith patch can be made as follows. Cannabis oil or one or more pure cannabinoids can be combined with permeation enhancer only, combined with carrier only, or combined with both permeation enhancer and carrier. Carrier can comprise, for example, one or more of oleic acid and dodecylmethyl sulfoxide. Then one or more pure terpenes, or an essential oil, or a combination of an essential oil and one or more pure terpenes, is mixed with the above combination. Then, a polymer such as a polyisobutylene polymer is mixed in. Finally, the mixture is spread into one or more sheets, cured at room temperature for several hours or longer. After drying, a foam backing layer is applied, and then the product is cut into shapes (e.g., squares, rectangles, ovals, round-edged squares or round-edged rectangles, circles) suitable for applying to the skin of a person.

In embodiments, the present disclosure provides permeation enhancers (“enhancers”),

A laminate that can be held in place on the gingiva (gums) takes the form of a semipermeable outer layer, reservoir having a pharmaceutical, backing layer, where the backing layer faces the gingiva. Saliva can enter through the semipermeable outer layer, pass through the reservoir, and then draw medicine into contact with gingiva for absorption in the bloodstream. A pharmaceutical can be freeze dried or can occur as a hydrogel matrix, in the reservoir. The present disclosure provides a backing layer of one or more polymers, such as, ethyl cellulose, butyl cellulose, hydroxybutyl cellulose, or polyvinylalcohol. An amorphous or semi-crystalline excipient matrix can be made from methylcellulose, ethylcellulose, hydroxypropyl methylcellulose, cellulose acetate phthalate, or cellulose acetate butyrate. In exclusionary embodiments, the present disclosure can exclude one or more of these polymers.

In reservoir-distribution embodiments, a pharmaceutical or nutraceutical can be distributed evenly throughout reservoir or can be distributed at a higher concentration at center of reservior, or can be distributed at a higher concentration at region of reservoir that is closer to the skin when patch is situated and adhering to skin.

Tackifiers

The present disclosure provides compositions, patches, and methods, that encompass one or more of Escorez 1000 Series-aliphatic resins; Escorez 2000 Series-aromatic modified aliphatic resins; Escorez 5300 Series-water white hydrogenated cycloaliphatic resins; Escorez 5400 Series-light color hydrogenated cycloaliphatic resins; Escorez 5600 Series-light color hydrogenated aromatic modified cycloaliphatic resins; Escorene® Ultra ethylene vinyl acetate (EVA) copolymers; ExxonMobil® ethylene n-butyl acrylate (EnBA) copolymers; Optema® EMA (ethyl methyl acrylate) resins (ExxonMobil, Inc.).

Escorez® 5400 is a hydrocarbon polymer additive available from ExxonMobil Chemical Company. It has a softening point of 103° C., a weight average molecular weight of about 400 g/mole, and a dicyclopentadiene/cyclopentadiene/methylcyclopentadiene content of 40-80 wt % (see, WO2013/176712 of Block).

Escorez® 5415 is a hydrocarbon polymer additive available from ExxonMobil Chemical Company. It has a softening point of 118° C., a weight average molecular weight of about 430 g/mole, and a dicyclopentadiene/cyclopentadiene/methylcyclopentadiene content of 40-80 wt % (see, WO2013/176712 of Block).

Escorez® 5340 is a hydrocarbon polymer additive available from ExxonMobil Chemical Company. It has a softening point of 140° C., a weight average molecular weight of about 460 g/mole, and a dicyclopentadiene/cyclopentadiene/methylcyclopentadiene content of 40-80 wt % (see, WO2013/176712 of Block).

Escorez® 5600 is a hydrocarbon polymer additive available from ExxonMobil Chemical Company. It has a softening point of 103° C., a weight average molecular weight of about 520 g/mole, and a dicyclopentadiene/cyclopentadiene/methylcyclopentadiene content of 40-80 wt % (see, WO2013/176712 of Block).

Escorez® 5615 is a hydrocarbon polymer additive available from ExxonMobil Chemical Company. It has a softening point of 118° C., a weight average molecular weight of about 500 g/mole, and a dicyclopentadiene/cyclopentadiene/methylcyclopentadiene content of 40-80 wt % (see, WO2013/176712 of Block).

In exclusionary embodiments, composition, medical device, dermal patch, and related methods of the present disclosure can exclude any composition, medical device, dermal patch, and method that comprises any one or of the above chemicals.

Hydrogels

Hydrogels are 3-dimensional, cross-linked networks of water-soluble polymers. The porous structure of hydrogels can be altered by changing the density of cross-linking. The degree of cross-linking can alter the rate of loading a drug, and it can alter the rate of drug release. The present disclosure can encompass a hydrogel that consists of one of the following polymers or alternatively, that comprises one or more of the following polymers (e.g., as a block polymer). The polymers include, poly(ethylene oxide) (PEO), poly(propylene oxide) (PPO), poly(lactide-co-glycolic acid) (PLGA), poly(N-isopropylacrylamide) (PNIPAM), poly(propylene fumarate) (PPF), poly(caprolactone) (PCL), poly(urethane) (PU), and poly(organophosphazene) (POP). An example of a block polymer is PEO-PPO-PEO. In exclusionary embodiments, the present disclosure can exclude a hydrogel that includes PEO, PPO, PLGA, PNIPAM, PPF, PCL, PU or POP. The present disclosure also encompasses hydrogels that contain a cyclodextrin, where the cyclodextrin is cross-linked to hydrogel (see, Hoare et al (2008) Hydrogels in drug delivery: Progress and challenges. Polymer. 49:1993-2007). Hydrogels of the present disclosure can be ethylene vinylacetate, alginic acid, gums, polyvinylalcohol hydrogel; silicone hydrogel; polyvinylalcohol/dextran hydrogel; alginate hydrogel; alginate-pyrrole hydrogel; gelatin/chitosan hydrogel; polyacrylic acid hydrogel; photo crosslinked polyacrylic acid hydrogel; amidated pectin hydrogel; pectin hydrogel; gelatin hydrogel; polyethylene glycol (PEG) hydrogel; carboxymethylcellulose/gelatin hydrogel; chitosan hydrogel, as well as mixtures thereof, or copolymers thereof, and the like. Hydrogels with crosslinks are available (Lee et al (2003) Eur. J. Pharm. Biopharm. 56:407-412).

In exclusionary embodiments, composition, medical device, dermal patch, and related methods of the present disclosure can exclude any composition, medical device, dermal patch, and method that comprises any one or of the above chemicals.

Printing Active Ingredients and Excipients on Dried Hydrogels

Dried hydrogel can take the form of a “xerogel” or of a film. Xerogel can be made by freeze drying a hydrogel. Film can be made by evaporative drying or casting from organic solutions. Spotting device can be used to apply microdrops in predetermined locations of dried hydrogel or on a film (see, e.g., U.S. Pat. No. 6,642,054 of Schermer). Where dried hydrogel or film takes the form of a layer, microdrops can be applied to one side only or to both sides. Where more than one type of drug is to be applied and where at least two of the drugs are incompatible with each other, or where a drug and an excipient are to be applied, and where these are incompatible with each other, these can be applied at different locations on the dried hydrogel or on the film. Drop size of microdrops can be, e.g., 0.05 nanoliters (nL)-10,000 nL, 0.5 nL-200 nL, 10 nL-100 nL, and so on. Drug, active ingredient, and/or excipient is not incorporated into the dried hydrogel, but is instead printed on its surface or surfaces. Printing on dried hydrogel avoids problems arising from incompatibility of drug, active ingredient, and/or excipient with the hydrogel itself. See, US2008/0095848 of Stabenau, which is incorporated by reference in its entirety.

Cyclodextrins

Cyclodextrins are cyclic oligosaccharides of (alpha-1,4)-linked alpha-D-glucopyranose units, with a lipophilic central cavity and a hydrophilic outer surface. As a result of their molecular structure and shape, they can act as molecular containers by trapping drugs or other molecules in their internal cavity. No covalent bonds are formed or broken during drug cyclodextrin complex formation, and in aqueous solution, the complexes readily dissociate, and free drug molecules remain in equilibrium with the molecules bound within the cyclodextrin cavity (see, Tiwari et al (2010) Cyclodextrins in delivery systems: Applications. J. Pharm. Bioallied Sci. 2:72-79). Derivatives of cyclodextrins that are hydroxypropyl (HP), methyl (M) and sulfobutylether (SBE) substituents are useful as pharmaceutical excipients.

Cyclodextrins for use, for example, in cannabinoid/cyclodextrin complex, include beta-cyclodextrins such as hydroxypropyl-beta-cyclodextrin, sulfobutylether-beta-cyclodextrin, maltoxyl-beta-cyclodextrin, and methylated cyclodextrins. Encompassed are alpha-cyclodextrins (6 glucopyranose units), beta-cyclodextrins (7 glucopyranose units), and gamma-cyclodextrins (8 glucopyranose units). Methylated cyclodextrins can improve aqueous solubility, dissolution rate, and bioavailability of cannabinoids.

The present disclosure provides a dermal patch (or buccal patch) comprising a dextrin where the dextrin is not complexed with a pharmaceutical agent, and a dermal patch (or buccal patch) comprising a dextrin where the dextrin is, in fact, complexed with a pharmaceutical agent.

In exclusionary embodiments, the present disclosure can exclude a formulation that comprises a cyclodextrin, or that comprises an alpha-cyclodextrin, or that comprises a beta-cyclodextrin, or that comprises a gamma-cyclodextrin. What can also be excluded is a device that comprises a cyclodextrin, such as an adhesive dermal patch comprising a dextrin or a buccal patch comprising a dextrin. Also, in exclusionary embodiments, composition, medical device, dermal patch, and related methods of the present disclosure can exclude any composition, medical device, dermal patch, and method that comprises any one or of the above chemicals.

Matrices, Carriers, Binders, Manufacturing Methods

A matrix, carrier, or binder, can include, e.g., hydrogel, polyethylene oxide, polyvinylpyrrolidone, hydroxypropyl cellulose, ethyl cellulose, methylcellulthose, alkylcelluloses, veegums clays, alginates, PVP, alginic acid, carboxymethylcellulose calcium, microcrystalline cellulose, polacrillin potassium, sodium alginate, corn starch, potato starch, pregelatinized starch, corn starch, modified starch, carnuba wax, montmorrilonite clays such as bentonite, gums, shellac, agar, locust bean gum, gum karaya, pecitin, tragacanth, and the like. In exclusionary embodiments, what can be excluded is one or more of the above polymers, clays, waxes, hydrogels, starches, and gums. A polyol can be used, for example, as a carrier. Polyols include propylene glycol and glycerol and the preferred (poly) alkoxy derivatives include polyalkoxy alcohols, in particular 2-(2-ethoxyethoxy) ethanol (Transcutol®).

In exclusionary embodiments, composition, medical device, dermal patch, and related methods of the present disclosure can exclude any composition, medical device, dermal patch, and method that comprises any one or of the above chemicals.

Matrix can be manufactured by melt-granulation, melt-extrusion, using particulates, granules, bilayers, plasticizers, and the like (see, US2016/0151502 of Wright). Patch can be made with silicone adhesives disposed on a substrate, copolymers, block polymers, tackifying resins, hot melt coating processes (see, US2014/0349108 of Fung). Patch can be made with backings, release liner, pressure sensitive adhesives, silicone gel adhesives (see, US2014/0287642 of Kumar). Dermal patch, buccal patch, tablets, can be made with excipient, disintegrant, swelling agent, films, binders, and the like (US2014/0079740 of Salama). Each of these patent documents is incorporated herein by reference in its entirety. Hot-melt extrusion, granules, tablets, transmucosal patches, transdermal patches, and methods of manufacture are detailed (Crowley et al (2007) Drug Development Industrial Pharmacy. 33:909-926; Repka et al (2007) Drug Development Industrial Pharmacy. 33:1043-1057).

Enhancers

Compositions, medical devices, dermal patches, and related methods of the present disclosure can comprise one or more of the following enhancers and solvents: dihydromyricetin, diethylene glycol monoethyl ether (Transcutol®), triacetin, dipropylene glycol, isophytol, phytol, oleic acid, a terpene, ethanol, azone (azone is “1-dodecyl azepan-2-one”), oleic acid, dimethylsulfoxide (DMSO), oleic acid, and limonene.

In exclusionary embodiments, composition, medical device, dermal patch, and related methods of the present disclosure can exclude any composition, medical device, dermal patch, and method that comprises any one or more of the above chemicals.

The present disclosure provides permeation enhancers, for example, for use with a dermal patch or for a buccal patch. Suitable permeation enhancers include, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides (see, Shojaei et al (June 2001) Systemic drug delivery via the buccal mucosal route. Pharmaceutical Technology. Pages 70-81). Other enhancers of the present disclosure are 1-octanol, 2-ethylhexanol, 1-nonanol, 1-decanol, and so on.

In exclusionary embodiments, compositions, formulations, dermal patch comprising one or more of said compositions or formulations, medical device comprising one or more of said compositions or formulations, can exclude any composition, formulation, dermal patch, medical device, or related method, that comprises one or more of the above-disclosed chemicals.

Permation enhancers of the present disclosure can be a biphasic composition having a lipid phase and a water phase. Lipid phase can be prepared by mixing isopropyl palmitate and lecithin. Water phase can be mixture of water and a surfactant. Surfactant can be Pluronic®, Pemulen®, Noveon®, or Carbopol®. Pemulen polymeric emulsifiers are high molecular weight, copolymers of acrylic acid and C10-C30 alkyl acrylate crosslinked with allyl pentaerythritol (Lubrizol, Inc. product sheet). Carbopol homopolymers are acrylic acid crosslinked with allyl sucrose or allyl pentaerythritol. Carbopol copolymer are acrylic acid and C10-C30 alkyl acrylate crosslinked with allyl pentaerythritol (Lubrizol, Inc. product sheet). Noveon® Polycarbophil, USP is a high molecular weight acrylic acid polymer crosslinked with divinyl glycol (Lubrizol, Inc. product sheet). Pluronic® polymers are block copolymers based on ethylene oxide and propylene oxide. They can function as antifoaming agents, wetting agents, dispersants, thickeners, and emulsifiers (BASF, Inc. product sheet). The present disclosure can exclude any formulation, composition, device, method, and such, that comprise one or more the molecules found in Pluronic®, Pemulen®, Noveon®, and Carbopol®.

PLOGel is “Pluronic Lecithin Organogel” (Pharmedica Enterprise, Selangor, Malaysia). PLOGel takes the form of an aqueous phase (240 mL poloxamer 407, potassium sorbate, water) and organic phase (60 mL lecithin, isopropyl palmitate, sorbic acid). The present disclosure can exclude any formulation, composition, device, method, and such, that comprise one or more of PLOGel, poloxamer 407, potassium sorbate, isopropyl palmitate, sorbic acid, lecithin, and the like.

In exclusionary embodiments, any composition, medical device, dermal patch, and related method of the present disclosure can exclude any formulation, composition, device, method, and such, that encompasses one of the above polymers, polymer compounds, and crosslinked polymer compositions.

In other exclusionary embodiments, the present disclosure can exclude compositions, formulations, dermal patches, layers, and the like, as well as methods, that comprise sulphoxides such as DMSO, Azones and Azone analogs such as laurocapram, transkarbams, 6-aminohexane acid esters, and can also exclude pyrrolidones such as 2-pyrrolidone, alcohols such as ethanol or decanol, glycols such as propylene glycol, surfactants, or vesicular carriers such as liposomes (see, Bartosova and Bajgar (2012) Curr. Med. Chem. 19:4671-4677).

Other chemicals that can be included in compositinos, dermal patches, and medical devices of the present disclosure, or that can be excluded from compositions, dermal patches, and medical devices of the present disclosure. The present disclosure provides camphor, zucapsaicin, silk fibroin, nonivamide, nicoboxil, davasaicin, piperine, rutaecarpine, capsaicinoids, resiniferotoxin, 3-hydroxyacetanilide, anandamide, Boswellia serrata extract, alpha-acaridial, benzoin resin, beta-acaridial, pseudocapsaicin, astaxanthin, dihydrocapsaicin, nordihydrocapsaicin anandamide, zingerone, warburganal, polygodial, aframodial, cinnamodial, cinnamosmolide, methylsufonlymethane, cinnamolide, isovelleral, methyl salicilate, scalaradial, ancistrodial, olvanil, merulidial, scutigeral, civamide, N-arachidonoyldopamine, glucosamine, eugenol, guaiacol, vanillotoxins, benzocaine, resiniferatoxin (RTX), hydrocortisone, salicylic acid, and triethanolamine salicilate.

In exclusionary embodiments, compositions, formulations, dermal patch comprising one or more of said compositions or formulations, medical device comprising one or more of said compositions or formulations, can exclude any composition, formulation, dermal patch, medical device, or related method, that comprises one or more of the above-disclosed chemicals.

Apertures and Pores

The present disclosure can encompass films, sheets, layers, membranes, and the like, including those with a plurality of apertures or pores. In some aspects, the apertures or pores have an average diameter of 20 nm, 40 nm, 50 nm, 100 nm, 200 nm, 300 nm, 400 nm, 500 nm, 600 nm, 800 nm, 0.001 mm, 0.002, 0.005 mm, 0.010 mm, 0.015 mm, 0.020 mm, 0.025 mm, 0.030 mm, 0.040 mm, 0.050 mm, 0.075 mm, 0.10 mm, 0.20 mm, 0.30 mm, 0.40 mm, 0.50 mm, and the like. Also, the pores can have a diameter range where the range is bracketed by any two of these values. In other aspects, the apertures or pores have a diameter in the range of 20-40 nm, 40-60 nm, 60-80 nm, 50-100 nm, 100-200 nm, 200-400 nm, 400-600 nm, 600-800 nm, 800-1,000 nm, 0.001-0.002 mm, 0.001-0.005 mm, 0.005-0.010 mm, 0.010-0.020 mm, 0.020-0.040 mm, 0.025-0.050 mm, 0.050-0.075 mm, 0.075-0.10 mm, 0.10-0.20 mm, 0.20 mm-0.40 mm, 0.25-0.50 mm, 0.50-0.75 mm, 0.50-1.00 mm, 0.1-0.2 mm, and so on. In exclusionary embodiments, the present disclosure can exclude films, sheets, layers, and the like, that have apertures or pores having any of the above average values, or that are describable by any of the above ranges.

Porous membranes can take the form of hydrophilic porous membranes and hydrophobic porous membranes, without implying any limitation. Hydrophobic membranes, such as hydrophobic polyethylene (PE) membranes, can be made more hydrophilic by alcohol or surfactants (see, WO2010/072233 of Calis). Pores in membranes of the present disclosure can have an average diameter of about 5 micrometers, about 10, about 15, about 20, about 25, about 30, about 40, about 50, about 60, about 70, about 80, about 90, about 100, about 110, about 120, about 130, about 140, about 150, about 160, about 170, about 180, about 190, or about 200 micrometers, and the like. Also, pores in the membranes can have an average diameter somewhere in the range 5-20 micrometers, 20-40 micrometers, 40-60 micrometers, 60-80 micrometers, 80-100 micrometers, 100-120 micrometers, 120-140 micrometers, 140-160 micrometers, 160-180 micrometers, 180-200 micrometers, and so on. In exclusionary embodiments, the present disclosure can exclude any membrane that is characterized by one of the above “about” values or that is characterizable by one of the above ranges.

For any given film, sheet, or layer, and the like, the area of a plurality of apertures or the area of a plurality of pores can occupy about 1%, about 2%, about 4%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, and the like of the surface area. In exclusionary embodiments, the present disclosure can exclude any film, sheet, or layer, where the area does not occupy one or more of the given percentage values, or where the area does not occupy a range between any two of the above given percentage values. The above parameters also can apply to a film, sheet, or layer, with perforations, where the value of the area for the perforation is measured flush with a surface of the film, sheet, or layer.

Solubilizers and Surfactants

Solubilizers such as detergents, surfactants, organic solvents, and chaotropic agents, are available for the present disclosure. These can be one or more of, polyethylene glycol (PEG), propylene glycol, dibutyl subacetate, glycerol, diethyl phthalate (phthalate esters), triacetin, citrate esters-triethyl citrate, acetyltriethyl citrate, tributyl citrate, acetyltributyl citrate, benzyl benzoate, sorbitol, xylitol, bis(2-ethyllhexyl) adipate, mineral oil, polyhydric alcohols such as glycerin and sorbitol, glycerol esters such as glycerol, triacetate; fatty acid triglycerides, polyoxyethylene sorbitan, fatty acid esters such as TWEENS, polyoxyethylene monoalkyl ethers such as BRIJ series and MYRJ series, sucrose monoesters, lanolin esters, lanolin ethers. These are available from Sigma-Aldrich, St. Louis, Mo. In exclusionary embodiments, what can be excluded is any composition, formulation, dermal patch, and methods that comprise one or more of these solubilizers or surfactants.

The present disclosure can encompass compositions, formulations, devices, and methods, that comprise one or more surfactants, such as, sorbitan trioleate, sorbitan mono-oleate, sorbitan monolaurate, polyoxyethylene (20) sorbitan monolaurate, polyoxyethylene (20) sorbitan monooleate, oleyl polyoxytheylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, isopropyl palmitate, glyceryl monooleate, glyceryl monostearate, glyceryl monoricinoleate, cetyl alcohol, stearyl alcohol, cetyl pyridinium chloride, olive oil, glyceryl monolaurate, corn oil, cotton seed oil, and sunflower seed oil.

In exclusionary embodiments, composition, medical device, dermal patch, and related methods of the present disclosure can exclude any composition, medical device, dermal patch, and method that comprises any one or more of the above chemicals.

Buffers and pH Values

The present disclosure can include formulations that contain a buffer with a pKa, as measured at room temperature, such as boric acid (pKa 9.2), CHES (pKa 9.5), bicine (pKa 8.3), HEPES (pKa 7.5), MES (pKa 6.1), MOPS (pKa 7.2), PIPES (pKa 6.8), Tris (pKa 8.1), imidazole (pKa 6.9), glycine (pKa 2.3), acetate (pKa 4.7), citrate (pKa 6.4), phosphate (pKa 7.21, 2.16, 12.32), malate (pKa 5.13), cacodylate (pKa 6.27), and the like.

In exclusionary embodiments, composition, medical device, dermal patch, and related methods of the present disclosure can exclude any composition, medical device, dermal patch, and method that comprises any one or more of the above chemicals.

Franz Diffusion Cell for Assessing Drug Release (Release from Monolithic Patches, Release from Reservoir Patches).

Franz diffusion cell is used to measure drug release kinetics from monolithic patches and from reservoir patches. Franz diffusion cells are described (see, Cavallari et al (2013) Eur. J. Pharm. Biopharm. 83:405-414; Franz (1968) On the diffusion of tritiated water through skin. J. Invest. Dermatol. 50:260; Balazs, Sipos, Danciu (2015) Biomedical Optics Express. 7:67-78; Simon et al (2016) Int. J. Pharmaceutics. 512:234-241; Jung et al (2016) Int. J. Cosmet. Sci. 38:646-650; Technical Brief 2009, Vol. 10, Development and Validation In Vitro Release Testing Methods for Semisolid Formulations, Particle Sciences, Bethlehem, Pa.). Franz diffusion cells and equipment for transdermal diffusion testing are available (Teledyne Hanson Research, Chatsworth, La.).

The present inventor uses a Franz cell for assessing release kinetics, as described below. For testing release kinetics from transdermal monolithic patches, where a semi-solid adhesive matrix is used, the Franz cell has the following components, from top to bottom: (1) Stopper used to seal top of donor compartment; (2) Donor compartment; (3) Patch situated at very bottom of donor compartment, with adhesive side of patch attached to human cadaver skin; (4) Human cadaver skin located immediately under the patch; (5) Receiving compartment, located immediately below the skin. Receiving compartment is filled with ethanol/water solution; (6) Magnetic stirrer located at bottom of receiving compartment.

For testing release kinetics from transdermal reservoir patches, the Franz cell has the following components, from top to bottom: (1) Stopper used to seal top of donor compartment; (2) Donor compartment; (3) Cream or gel in the donor compartment; (4) Microporous membrane (Solupor® from Lydall Performance Materials, Inc., Rochester, N.H.); (5) Human cadaver skin located under the microporous membrane; (6) Receiving compartment is filled with phosphate buffered saline at pH 6; (7) Magnetic stirrer at bottom of receiving compartment.

Receiving Solutions for the Franz Cell Used to Test Patches of the Present Disclosure

This concerns receiving solutions that were used for testing the monolithic patches and dermal patches of the present disclosure. The receiving solution can be a saline solution for drugs that are soluble in water or alcohol-water solutions for drugs that are not well soluble in saline.

According to Bartosova and Bajgar (2012) Current Medicinal Chemistry. 19:4671-4677, dermal absorption involves these steps: (1) Penetration. Entry of a substance into a particular layer of the skin, such as the layer that is “stratum corneum;” (2) Permeation. This is penetration through one layer into another slayer, where the layers differ both structurally and functionally from each other; (3) Absorption. Uptake of substance into the lymphatics or into the bloodstream. The stratum corneum is lipophilic while, in contrast, the epidermis and dermis are hydrophilic. Thus, lipophilicmolecules may pass at a greater rate through stratum corneum while, in contrast, hydrophilic molecules may pass at a greater rate through epidermis and dermis. Rate of transfer can be expressed by Frick's law: Jₛₛ=(Kₚ)(Cₒ). Jₛₛ is steady state flux per unit area. Kₚ is permeability coefficient for a given solute in a given vehicle (centimeters per hour). Cₒ is concentration of solute in the donor compartment. Kₚ predicts the penetration rate of a chemical at a given concentration from the same vehicle. Kₚ is independent of concentration and time.

According to Bartosova and Bajgar, supra, guidance for in vitro skin absorption tests is available from OECD (2004) OECD Guideline for the Testing of Chemicals. Skin Absorption: in vitro method. Pages 1-8). Diffusion dells are commonly used to measure in vitro skin absorption, and these can be of the static type or flow-through type. The Franz diffusion cell has the following structures, in order from top to bottom: Donor compartment (containing test substance, such as a drug); Membrane (supporting membrane where skin is positioned); Receptor compartment with sampling port that allows access to receptor compartment; Surrounding lower half of receptor compartment is water jacket for maintaining temperature; At bottom of interior of receptor compartment, and in contact with fluid in receptor compartment, is magnetic stirrer. Optionally, the researcher can include dermal absorption tests with standards, such as benzoic acid, caffeine, and testosterone.

Also, according to Bartosova and Bajgar, supra, dose concentrations up to 10 mg/cm² or up to 10 microliters/cm² are used. The skin sample is equilibrated with receptor fluid for 10-30 minutes before applying dose to skin. Barrier integrity of skin is checked by methods that determine transepidermal water loss or transcutaneous electrical resistance. Kinetic parameters that can be determined include flux (J), permeability coefficient (Kr), and diffusion coefficient (D). When the testing period comes to its scheduled end, for example, after three hours or after 24 hours, some of the test substance may still located inside the skin, that is, in the membranes and cytosol of skin cells. Test substance inside the skin may optionally be included in the value for total substance that is absorbed.

Source of Human Skin

For CBD and THC, the inventor used for the receiving solution ethanol/water mix (30/70 by weight). The cadaver skin, the inventor received skin from a tissue bank such as Science Care in Phoenix Ariz. The donor human skin was dermatomed in Science Care to thickness of about 250 micrometers consisting of stratum corneum and part of epidermal layer and shipped to the inventor on dry ice.

After receiving the donor human skin, the inventor prepared the skin for testing in a Franz cell by thawing the skin to room temperature, washing in distilled water and cutting in round pieces to fit the diameter of the Franz cell opening. Before placing the patch on the human skin, the inventor dried the skin with a paper tissue. The skin with the attached patch was placed between the Upper Donor Chamber and the Lower Receiving Chamber and clamped tightly. After that, the receiving solution was filled into the Receiving Chamber making sure no bubbles are trapped beneath the skin. The test lasts typically 24 hrs and aliquots of 150 mL were withdrawn from the receiving chamber after different periods of time and analyze on HPLC. Display of the drug concentration in the receiving solution versus time was presented by graphs showing the kinetics of the transdermal passage of the drug from the patch through the skin into the receiving solution.

Measuring Thickness of Films and of Patches

Film thickness can be measured using puncture test and texture analyzer, such as Instron® 3366-2716015, Germany (see, Priya et al (2011) J. Pharm. Res. 3:56-65).

Patch thickness can be measured with a screw gauge, where thickness can be measured at various spots on the patch. To measure surface pH, patch can be allowed to swell for 2 hours on the surface of an agar plate (2% w/v), and the pH then measured with pH paper. Swelling can be measured by taking the weight each hour for six hours, after placing patch on an agar plate (see, Verma et al (2014) Effect of novel mucoadhesive buccal patches of carvediol on isopenaline-induced tachycardia. J. Adv. Pharm. Technol. Res. 5:96-103). Residence time measured time that patch adheres to a mucosal membrane, where patch is glued to a substrate, with repeated up-and-down movement of the substrate until the patch detaches (see, Ismail et al (2003) Design and characteristics of mucoadhesive buccal patches containing cetyl pyridinium chloride. Acta Pharm. 53:199-212.

Dimple-Style Reservoir Vs. Balloon-Style Reservoir for Reservoir Patch Device

In a balloon embodiment, the present disclose can include a reservoir that is conformed like a sealed bag (or like a continual bag) or like a sealed balloon. In this embodiment, the reservoir is made of a material that is separate from backing and separate from permeable layer. In this embodiment, the reservoir may or may not be attached to backing or permeable layer by way of an adhesive or heat seal.

In a dimple embodiment, the reservoir has on a distal side a backing that has a dimple (or outpouching) where the dimple is conformed to hold drug, and where the reservoir has on proximal side a permeable layer. In other words, what prevents drug from spillout out of the outpouching is this permeable layer.

The backing and permeable layer are attached to each other, to prevent leaking of the drug. Attachment can be via an adhesive or heat-sealing. The present disclosure can exclude devices where this attachment is by adhesive and can exclude devices where this attachment is by heat-sealing.

The present disclose can exclude devices with balloon reservoir. In other embodiments, the present disclosure can exclude devices with a dimple reservoir.

In the dimple embodiment, the permeable layer can comprise a plurality of slits, a plurality of tiny holes, or by being made of a porous layer. The present disclosure can exclude device with dimple reservoir.

Dimple reservoir device can include (or exclude) a layer that that resides in between drug and permeable layer. Also, dimple reservoir device can include (or exclude) a layer that resides on side of permeable layer facing the skin, where this layer is in substantial contact with the permeable layer. This layer that is on side of permeable layer of skin can be distal to adhesive layer and peelable backing layer.

Permeable layer can comprise permeable polypropylene film (US2006/0024520; US20016/115585), permeable polyethylene film (U.S. Pat. No. 4,793,003; WO2006/070672); permeable polyurethane film (U.S. Pat. No. 9,566,423).

Shapes of Reservoirs that Alter Delivery Rate Over the Course of Time

Reservoir of the present disclosure can be manufactured in predetermine shape, so that rate of release of an active agent to the skin or to a mucosal surface various over the course of hours, during the time frame when patch device is worn by a patient. For example, reservoir can be conical, where the wide surface (base of cone) is situated at the distal portion of patch device and where point of the cone is situated at the proximal portion of patch device. Proximal means the side of patch device closest to the skin, distal means the side of patch device farthest away from the skin. With cone reservoir, rate of drug transfer from patch to skin or mucosal surface gradually decreases over time. Reservoir can be hemispherical, resembling a gum drop, with base of gum drop closest to proximal side of patch device, and rounded surface of gum drop closest to distal side of patch device. Hemispherical reservoir gives initial rapid rate of drug release followed by rapid decrease in rate of drug release. Reservoir can also have edges that are perpendicular to the skin-facing portion of the patch, that is, perpendicular to the peelable release of the dermal patch (in the event that the patch has a peelable release). See, U.S. Pat. No. 6,207,181 of Herrman, which is incorporated herein by reference in its entirety. The present disclosure can exclude a device with conical reservoir, with hemispherical reservoir, and/or hemispherical reservoir.

The reservoir device of the present disclosure can have only one conical reservoir, only two conical reservoirs, only three conical reservoirs, at least one conical reservoir, at least two conical reservoirs, at least three conical reservoirs. The reservoir device can have only one, only two, only three, at least one, at least two, at least three hemispherical reservoirs. The reservoir device can have only one, only two, only three, at least one, at least two, at least three perpendicular sided reservoirs. Moreover, the reservoir device can have only conical reservoir(s), only hemispherical reservoir(s), only parallel sided reservoir(s), a combination of only conical reservoir(s) and hemispherical reservoir(s), a combination of only conical reservoir(s) and parallel side reservoir(s), a combination of only hemispherical and parallel side reservoir(s), or a combination of all three of conical, parallel side, and hemispherical reservoirs. The present disclosure also provides reservoirs of an ambiguous shape, such as that resembling a wrinkled, partially filled balloon, either alone or in combination with a conical, hemispherical, and/or parallel shaped reservoir. The present disclosure also provides reservoirs having the shape of a hot dog, either alone or in combination with a conical, hemispherical, and/or parallel shaped reservoir.

Emulsions and Self-Emulsifying Agents

The present disclosure provides emulsions, emulsifying agents, self-emulsifying agents, creams, and lotions. The following provides examples of self-emulsifying agents. Self-emulsifying drug delivery systems (SEDDS) and self-nano-emulsifying drug delivery systems (SNEDDS) have been reviewed (see, Cherniakov et al (2015) Expert Opin. Drug Deliv. 12:1121-1133). Self-emulsifying agents include glycerol monostearate, glycerol monooleate, and Cremophor RH40®. Cremophor RH40® is polyoxyl 40 hydrogenated castor oil. Cremophor EL® is polyoxyl 35 castor oil. These chemicals can be obtained from BASF Aktiengesellschaft, Ludwigshafen, Germany. In one aspect, the present disclosure can include formulations that comprise a self-emulsifying agent. In another aspect, the present disclosure can exclude formulations, and can exclude devices, that comprise a self-emulsifying agent.

Solubilizer SL-11 is a self-emulsifying agent that provides a nanoemulsion suitable for containing a hydrophobic drug (NOF America Corp., Irvine, Calif.). Emulsion with particle size under 50 nanometers can be made by these steps: (1) Dissolve drug in a suitable solvent, such as ethanol; (2) Add the drug solution prepared in (1) to Solubilizer SL-11, thoroughly mix to completely dissolve the contents; (3) The drug/SL-11 solution with solvent is made; (4) Evaporate the solvent at 50 degrees for about 1 hour to remove the solvent, or remove the solvent under a nitrogen stream; (5) Concentrated solution of SL-11 and the drug is made; (6) Soft capsules can be prepared by using the concentrated solution in (5) (NOF America Corp., Irvine, Calif.).

Self-emulsifying agents can be made with polyglycolyzed glycerides (PGG) with varying fatty acid and polyethylene glycol (PEG) chain lengths, where these produce the self-emulsification of oil in water. The quality of the resulting emulsions depends on the oil and emulsifier pair selected and on the concentration of PGG as the emulsifier. One suitable oil is an oil with a medium-chain triglycerides (caprylic acid and capric acid; Neobee M5®). Another suitable oil is peanut oil. With formation of the emulsion, parameters that can be measured include droplet size distribution, droplet polarity, the release rate of the drug and the oil/water partition coefficient of the drug. PGG was found to be a workable emulsifier for use in self-emulsifying drug delivery systems (SEDDS) (Shah et al (1994) Int. J. Pharmaceutics. 106:15-23).

Yet another non-limiting example of a self-emulsifying agent is provided by Chambin et al (2004) Int. J. Pharmaceutics. 278:79-89. This describes a self-emulsifying system using Gelucire® 44/14, an excipient from the lauroyl macrogolglycerides family. The laboratory method involves producing a fine oil-in-water emulsion when introduced into an aqueous phase under gentle agitation as SEDDS. The advantage is improved solubility and bioavailability of poorly water-soluble drugs. Gelucire® 44/14 was ground into a powder by cryogenic grinding to produce solid oral dosage forms and resulting in formulations made of Gelucire® 44/14 and ketoprofen (90/10). Cryogenic grinding produced Gelucire® 44/14 in a powder form, where this process did not change its physical properties, emulsification capacities and dissolution performances of the formulation tested.

Devani (2004) J. Pharmacy Pharmacology. 56:307-316, provide the following example, using the drugs danazol and mefenamic acid. In self-emulsifying drug delivery systems (SEDDS), drugs are dispersed in an oil-surfactant mix that emulsifies on contact with water. Self-emulsifying systems can be based on the Labrafil family of polyglycolysed oils, using Tween 80 and Tween 20 as surfactants. The more hydrophilic oil-surfactant mixes showed a greater ease of emulsification and a lower particle size. A linear relationship was observed between the hydrophile-lipophile balance (HLB) of the mix and the solubility of both danazol and mefenamic acid, with more hydrophilic mixes showing greater drug solubility values.

This provides another non-limiting example. Zupancic et al (2016) Eur. J. Pharm. Biopharm. 109:113-121 described emulsifying properties of SEDDS composed of long chain lipids (LC-SEDDS), medium chain lipids (MC-SEDDS), short chain lipids (SC-SEDDS) and no lipids (NL-SEDDS). The drug, enoxaparin was incorporated via hydrophobic ion pairing in the chosen SEDDS. The average droplet size of chosen LC-SEDDS, MC-SEDDS and NL-SEDDS ranged between 30 and 40 nm. MC-SEEDS containing 30% Captex 8000, 30% Capmul MCM, 30% Cremophor EL and 10% propylene glycol and NL-SEDDS containing 31.5% Labrafil 1944, 22.5% Capmul PG-8, 9% propylene glycol, 27% Cremophor EL and 10% DMSO exhibited 2-fold higher mucus diffusion than LC-SEDDS. Both MC-SEDDS and NL-SEDDS showed sustained in vitro enoxaparin release. Orally administrated MC-SEDDS and NL-SEDDS yielded an absolute enoxaparin bioavailability of 2.02% and 2.25%, respectively.

Further regarding emulsions, emulsifying agent can be characterized by Hydrophilic Lipophic Balance (HLB). HLB system is numbered 1 to 20. HLB values of 3 to 6 are lipophilic and these form water-in-oil emulsions (see, Vadlamudi, Hyndavi, and Tejeswari (2014) Current Drug Discovery Technologies. 11:169-180). HLB values of 8 to 18 are hydrophilic and these form oil-in-water emulsions (see, Grimberg, Nagel, and Aitken (1995) Environ. Sci. Technol. 29:1480-1487).

Bioadhesive Materials

Bioadhesive polymer of the present disclosure, when swollen, creates a flexible network through with drug can diffuse. Bioadhesive material serves a matrix for retaining pharmaceutical agents, until patch is applied to the skin or to a mucosal surface of the consumer. Bioadhesive materials include, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene) (see, Shojaei et al (June 2001) Systemic drug delivery via the buccal mucosal route. Pharmaceutical Technology. Pages 70-81).

Dermal Patches with a Plurality of Adhesive Layers

The present disclosure provides dermal patches, laminated sheets, and related methods that comprise a plurality of adhesive layers. In one embodiment, a monolithic patch has these layers, from most distal to most proximal: (1) Backing; (2) Adhesive; (3) Carrier layer containing active agent; (4) Contact adhesive, and (5) Protective liner. In an exclusionary embodiment, the present disclosure can exclude this embodiment.

In another embodiment that is characterized by having a “rate controlling layer,” the monolithic patch has these layers, from distal to proximal: (1) Backing; (2) Adhesive; (3) Carrier layer containing active agent; (4) Adhesive layer; (5) Rate controlling polymer layer; (6) Adhesive layer; and (7) Protective liner. In an exclusionary embodiment, the present disclosure can exclude this embodiment.

The following concerns an embodiment where there is a “carrier layer” and where carrier layer is surrounded by and in contact with, on distal surface and on lateral surfaces, with an adhesive layer, and where carrier layer is surrounded by and in contact with, on proximal surface with “active ingredient permeable skin contact adhesive layer.” More generally, speaking present disclosure encompasses a “hat embodiment” taking the form of a dermal patch or other medical device where a first layer has a distal surface, proximal surface, and lateral surfaces. In this “hat embodiment” the distal surface, proximal surface, and lateral surfaces, are all surrounded by and in contact with a “hat layer.” The hat layer can be an adhesive layer, or it can be an impermeable backing layer. The term “hat embodiment” and “hat layer” refer to the fact that the “hat layer” covers the first layer, in the same way that a man's hat covers the top of his head as well as his ears, forehead, and back of his head. The present disclosure provides a device with these layers, from distal to proximal: (1) Backing; (2) Adhesive; (3) Carrier layer; (4) Active ingredient permeable skin contact adhesive; and (5) Protective liner. In this embodiment, the “hat” can cover the lateral sides of the carrier layer and also cover the lateral sides of the “active ingredient permeable skin contact adhesive layer.” In an exclusionary embodiment, the present disclosure can exclude the above “hat” embodiment.

In another “hat” embodiment, the present disclosure provides, from distal to proximal: (1) Backing; (2) Adhesive layer; (3) Carrier layer; (4) Active ingredient permeable layer; (5) Rate-controlling polymer layer; and (6) Active ingredient permeable skin contact layer. The “hat” takes the form of backing plus adhesive layer, and the had covers the laterals sides of all four of these layers: carrier layer, active ingredient permeable layer, rate-controlling polymer layer, and active ingredient permeable skin contact layer. In an exclusionary embodiment, the present disclosure can exclude the above “hat” embodiment.

In other exclusionary embodiments, the present disclosure can exclude devices where: (1) Carrier layer is in direct and substantial contact with backing layer; (2) Carrier layer is in direct and substantial contact with an adhesive layer; (3) Adhesive layer is in direct and substantial contact with rate-controlling polymer layer; (4) An adhesive layer is in direct and substantial contact with protective liner; (5) Where the device comprises a “hat” configuration of layers; (6) Carrier layer is in direct and substantial contact with active ingredient permeable layer, (7) Active ingredient permeable skin contact layer is in direct and substantial contact with protective layer; (8) Active ingredient permeable skin contact layer is in direct and substantial contact with release liner or protective liner; (9) Where at least part of device has “hat” configuration and where only one layer is covered (surrounded on proximal face and on lateral faces) by the hat; (10) Where at least part of device has “hat” configuration and where only two layers are covered (surrounded on proximal face and on lateral faces) by the hat; (11) Where at least part of device has “hat” configuration and where only three layers are covered (surrounded on proximal face and on lateral faces) by the hat; (12) Where at least part of device has “hat” configuration and where only four layers are covered (surrounded on proximal face and on lateral faces) by the hat. The exclusionary embodiments of the present disclosure encompass any combination of the above exclusions. The above may apply to reservoir patches where, optionally, “reservoir” takes the place of “carrier layer.” Also, the above may apply to monolith patches.

Manufacturing Processes and Equipment

Adhesive patches can be made by solvent casting or by direct milling. In solvent casting, all excipients and the drug are dispersed in an organic solvent and coated on a sheet of release liner. After solvent evaporates, a thin layer of protective material is laminated on the sheet of coated release liner to form a laminate. The laminate is then cut into patches (Koyi and Khan (2015) Buccal patches: A review. Int. J. Pharmaceutical Sciences Res. 4:83-89).

In direct milling, patches are created without using solvents. Drug and excipients are mixed by direct milling or by kneading, usually without any liquids present. After milling, the material is rolled on a release liner. A backing layer is then applied. Direct milling avoids the problem of residual solvents (Koyi and Khan (2015) Buccal patches: A review. Int. J. Pharmaceutical Sciences Res. 4:83-89).

The concerns solvent casting method and hot melt extrusion method. Buccal film can be made by solvent casting method and by hot melt extrusion method. Solvent casting involves dissolving water-soluble polymers to form viscous solution. Excipients are dissolved into solvent to give clear viscous solution. Then, both solutions are mixed (solution of water-soluble polymers; excipient solution) and then cast as a film, and then allowed to dry. This concerns hot melt extrusion. The drug or combination of drugs is in a dry state, and it is filled in a hopper, mixed, heated, and then extruded in a molten state. The molten mass that is formed is used to cast a film (Madhavi et al (2013) Buccal film drug delivery system—an innovative and emerging technology. J. Mol. Pharm. Org. Processing Res. Vol. 1, Issue 3 (6 pages)).

Sealing Two Strips Together at the Edges, and Coordinating Transverse Sealing to Create Pouches and Filling of the Pouches

What is provided is a method to feed two strips into a machine with rollers to move the strips at the same speed, and to cause the two strips to move downwards, where the first face of the first strip is caused to contact the first face of the second strip. The first face is caused to contact the first face of the second strip, in preparation for heating the edges of the two strips, thus sealing the two strips together, and in preparation for transverse heating, with heating at intervals of distance and time, thus creating a plurality of pockets in the sandwich of the two strips. When the two strips are moved downwards, the first strip and second strip are situated to form a thicker sandwich that moves downwards. Heaters resembling wheels or rollers, clamp down on the edges of the 2-strip sandwich, causing the 2-strip sandwich to form a long, closed tube. While the 2-strip sandwich moves downwards, what simultaneously occurs is simultaneous heating/sealing of a pair of transverse clamps. The transverse clamps create separate pouches in the long 2-strip sandwich. When the heated bars clamp down, what is created is a top seal of a previously filled pouch, and the bottom seal of a pouch that has yet to be filled. Simultaneously occurring with heating/sealing at the edges by the heated wheels, and simulataneously occurring with heating by the transverse bars, is filling of each pouch as it is created, where filling is by a long tube that reaches down into the long sandwich to fill each pouch as it is created. See, U.S. Pat. No. 6,871,477 of Tucker, which is incorporated by reference in its entirety. The first strip can comprise an adhesive layer and permeable membrane, the second strip can be an impermeable backing, and the gel can comprise a cannabinoid in gel form.

Unrolling Three Different Layers from Rolls, Stripping Off Release Liners from Two of the Layers, Aligning the Three Layers Together to Form a Complex, and Rolling the Complex on to a Roll

The present disclosure provides machinery that can unroll a plurality of rolls, optionally with stripping off a release-layer from one or more of the rolls, and taking up the stripped-off release-layer on an empty rotating drum or roll. For example, three different rolls can contain three different laminates, the first laminate comprising: (1) Protective backing; (2) Combined zone of transport enhancement and zone of containment; and (3) Release layer. The second laminate can comprise: (1) Adhesive layer; (2) Zone of transport control; and (3) Release liner. And the third laminate can comprise: (1) Support film; (2) Adhesive; and (3) Removable liner that is not removed during the above-mentioned method. Machinery can include three rolls on three rotating mechanisms of first, second, and third laminate, respectively. Machinery can include take-up rolls for taking up release liners. Machinery can include a pair of rollers situated on opposite sides of moving sandwich of first laminate and second laminate for use in bringing the two laminates together. Also, machinery can include a pair of rollers situated on opposite side of the nascent 3-part sandwich, where the 3-part sandwich takes the form of the combined (in contact with each other) first and second laminate and the entering third laminate. The entering third laminate is simultaneously unrolled from its roll and then combined with the complex of first and second laminate. The final product is then moved, by way of pairs of rollers situated on opposite sides of the moving final product, and also moved by way of individual rollers, e.g., rollers called over roller, under roll, and over idler roller. The above-disclosed machinery can also include a device for sealing laminates together, a corona discharge for enhancing the sealing of the laminates together, a device for depositing a drug or adhesive or other composition on one or more of the laminates as the laminate is unrolled from its roll, and cutting devices for separating the sandwich of three laminates into patches. See, U.S. Pat. No. 5,370,924, which is incorporated herein by reference in its entirety.

Layered device may be assembled and then sealed by vacuum forming or by heat sealing without vacuum. In exclusionary embodiment, the present disclosure can exclude machinery, methods, and patches, made using one or more of vacuum forming, heat sealing, corona discharge, one or more crimp rolls, or cooperating nip.

Providing a Platen with Bar-Like Regions Separated by Channels, and Using the Platen to Stamp a Laminate, and to Provide Pressure on Regions that Need to be Collapsed, while Refraining from Providing Pressure on Regions that Contain Drug and Matrix

The present disclosure provides machinery, such as a platen with bar-like regions separated by channels, and where the bar-like regions are optionally heated. The platen can be used to selectively compress parts of a laminate, where the laminate (the “workpiece”) comprises an upper layer that is a cellular region and a lower layer that is a skin adhesive. The platen selectively compresses the distal sides (the right edge and left edge), resulting in collapse of the distal sides of the laminate. Optionally, only the part of the laminate destined to be collapsed is provided with the adhesive. The cellular region can be reticulated or it can be non-reticulated. The cellular region can be made of foamed thermoplastic resin. Cell size can be about 0.05, about 0.1, about 0.2, about 0.4, about 0.6, about 0.80, about 1.0, about 1.2, about 1.4, about 1.6, about 1.8, about 2.0, about 2.5, about 3.0, or about 4.0 millimeters. Collapsed regions are such that drug cannot easily pass through collapsed regions. In embodiments, non-collapsed central area (the area that resided under the channel during platen-manufacturing process) can contain a distally-situated layer of drug-releasing matrix (which contains drug) in contact with a proximally-situated layer of a medium through which drug can diffuse. The layer of medium through which drug can diffuse can be, e.g., gel, cream, or ointment. “Distal” means away from the skin when patch is attached to skin, and “proximal” means on the side of patch that is closest to skin, when patch is attached to skin. The compressed lateral parts of patch may be called “straps.” See, U.S. Pat. No. 5,505,958 of Bello, which is incorporated herein by reference in its entirety.

In exclusionary embodiments, the present disclosure can exclude patch devices with non-compressed cellular region, patch devices with compressed cellular region, layered structures with a distally-situated drug matrix and a proximally-situated gel, cream, ointment, or other medium through which drug can diffuse on its way to skin. Also, the present disclosure can exclude any composition, laminate, layered structure, and patch that was made via heating of a layered structure or via heating of a laminate.

Placing Drug Between Two Webs, Sealing Two Webs Together, Crimping the Sealed Webs to Form Pockets, and Cutting the Sealed Web

The present disclosure provides machinery and methods for using, as starting material, two different webs, each on a roller, where each web comprises one or more of a film, adhesive layers, impermeable layers, porous layers, and the like. The finished product takes the form of the two webs that are sealed together, and where an active ingredient, such as a composition comprising one or both cannabinoid and terpene, is contained therein. In the method, a first supply roll provides one web and a second supply roll provides second web. Machinery at various “stations” modify one of the webs or modify both of the webs, as the webs move along a conveyor belt. One station, which is optional, is a corona discharge station. The corona discharge modifies the surface chemistry of one or both of the webs, prior to marriage of the two webs together by operation of two crimp rolls. The corona discharge modifies the surface chemistry to improve adhesive properties of the first web and/or of the second web. Corona discharge is preferred where dissimilar materials (one material of first web, and other material of second web) are to be adhesively joined. Dissimilar materials can be, e.g., polyester polymer and ethylene acrylic acid polymer.

Another station is deposit station, which deposits active substance on one of the webs, as the web moves towards the crimp roll. Deposit station can include a reservoir that contains drug and tube leading from reservoir to location on web surface where drug is to be deposited. The deposit station preferrably occurs after the corona station. Also, the deposit station and corona station preferrably act on the same web, though optionally deposit station can operate on first web and corona station can operate on second web. The two webs are securely fastened together in a station taking the form of a first crimp roll and a second crimp roll. These rolls resemble gears, in that first crimp roll has projections and second crimp roll has depressions, which act meshingly in the manner of a “tongue-and-groove” to compress the two webs together and, at the same time, to stamp the joined webs into a pocket-like shape. The regions of the first crimp roll and second crimp roll that mesh together are called a “cooperating nip.”

Finally, after the webs pass through the corona station, drug deposit station, and crimp rolls, the joined webs are cut by rotary die cutter, to create flexible packages or flexible patches suitable marketing. Motors can drive rollers. Also, motors can drive drimp rolls. See, U.S. Pat. No. 4,782,647 of Williams which is incorporated herein by reference.

Separating Cut Patches from a Strip of Non-Cut Patches, and Transferring Cut Patches to a Carrier

This describes only one step in procedure for making adhesive patches, e.g., monolithic devices and reservoir devices. The procedure involves a cutter, transfer devices resembling wedges, and rollers. The rollers function to move a first web and a second web, in the manner of a conveyer belt. The first web takes the form of an auxiliary layer film on top, and then just under it, a drug-containing adhesive layer that sits on top of a carrier film. The first web, which has these three layers, is then later on supplemented by a process layer, where the result is a web consisting of four layers (process layer on top, then auxiliary layer film, then drug-containing adhesive layer, and on the bottom, carrier film). An earlier-occurring cutting process has cut the auxiliary layer film and the drug-containing adhesive layer into blocks. The first web is moved in one direction, e.g., to the left, and then with the help of the transfer devices resembling wedges, the squares are separated from the carrier film (the carrier film is then moved away to the right) and also separated from the combination of auxiliary layer film and process layer (which is moved upwards), where the squares end up residing on a carrier film. At this point the blocks are separated from each other, and any scrap that had been created with the cutting process is then discarded. This refers to the situation where cutting creates discrete blocks and creates scraps in between the blocks. The supporting film supports the blocks and moves away to the left. See, U.S. Pat. No. 6,059,913 of Asmussen, which is incorporated herein in its entirety.

Cutting Laminate to Create Fully Cut-Out Region and, within it, a Partially Cut (Scored) Region

Machinery, methods, and workpiece of the present disclosure comprises sheet of laminate, where shapes of the sheet (rectangles, ovals, circles) are cut fully through the laminate, and where the edges of the cut-out laminate are called, “periphery” (outer cut). Where the cut-out laminate is circular, the periphery is the same as the circumferential region. In addition to being cut at the periphery, the sheet is simulataneously cut during the cutting operation in a region within the periphery (inner cut). The inner cut has a smaller diameter than the outer cut. Also, the inner cut is to a shorter depth than the outer cut. In the case of a 3-layer laminate (release layer; pressure-sensitive adhesive, backing), the outer cut slices through all three layers, but the inner cut slices only partially through the top layer (the release layer). This partial cutting is more properly called, “scoring” rather than “cutting.” The goal of this 2-distance cutting method is to score the release layer to facilitate easy removal of the liner by the user, and at the same time, to avoid leaking of adhesive from the patch during storage of the patch. Machinery for the 2-distance cutting method can take the form of a roller covered with cutting stampers (similar to cookie-cutters). Each cookie cutter stamps all the way through the laminate. Within each cookie cutter resides a second (smaller diameter) cookie cutter which is sized so that it only cuts partially through the top layer of the laminate (thus only scoring the top layer). In an alternative machinery, a first roller bears an array of only the larger diameter (and longer cutting distance) cookie cutters, while the second roller bears an array of the smaller diameter (and scoring distance) cookie cutters. In operation, the two rollers operate simultaneously, and the cookie cutters on the first roller are aligned exactly with the cookie cutters on the second roller and, in operation the cutting (cutting through all layers) occurs simultaneously with scoring, for each patch. See, U.S. Pat. No. 5,656,285 of Sablotsky, which is incorporated by reference in its entirety. In addition to the one cutting roller (or to the two cutting rollers), the machinery can have a pressure roller and a support roller, for use in driving the sheet of laminate. In exclusionary embodiments, the present disclosure can exclude an adhesive dermal patch that has a scored region, such as a scored release layer.

Efficient Separation of Punched Patches from Scrap Web

During manufacture of adhesive patches, patches are stamped out from, or cut from, a sheet consisting of various layers. These layers may include backing, matrix containing a drug, skin adhesive, and release layer. During cutting, some of the punched patches that have not yet been separated from scrap web may cling to the scrap web as the scrap web is pulled away from the sheet. Where this clinging is maintained as the scrap web is pulled away, the result will be undesirable discarding of the clinging punched patches along with the scrap web. This type of undesired clinging can be increased by flow of adhesive out of the edges of the punched patch, followed by flow of the adhesive to contact the scrap web. Efficient separation of punched patches can be accomplished by way of a probe or probes that contact the punched patch and shove the punched patch on to a horizontally moving conveyor belt as the scrap web is drawn upwards for eventual discard. The probe can take the form of a rotating roller where the roller is covered with blocks having the same shape and exactly the same dimensions (or dimensioned to be about 5% smaller, about 10% smaller, about 15% smaller, about 20 smaller, and the like, in area, as compared to the punched patch). The blocks can have a shape, as viewed from “above,” that is square, rectangular, oval, round, etc., and to have a shape corresponding to the punched patch. Thus, as the roller rotates, each block presses down on a corresponding punched patch (as the punched patch continues to move on the conveyor belt) while the scrap web is simultaneously detached and drawn upwards by the rotation of the roller. An alternative to using a roller covered with block probes, is a roller covered with flexible bristles. As the roller rotates, the bristles press springfully down on the punched patches, the bristles remaining bent, causing the punched patches to separate from the scrap web. At the same time, the bristles pressing on the scrap web are greatly bent at first, but as the scrap web is pulled upwards, the bristles spring out to their full (un-bent) length. See, US2017/0136648 of Grader, which is incorporated herein by reference in its entirety. In an exclusionary embodiment, the present disclosure can exclude manufacturing machinery and methods, comprising a roller with blocks or a roller with bristles, for use in preventing punched patches from adhering to the scrap web.

### EXCLUSIONARY EMBODIMENTS

The present disclosure can exclude a composition, formulation, dermal patch, methods of use, methods of manufacture, that comprise one or more of the following: capsaicin, 2-arachidonylglycerol, curcumin, glycerylmonooleate, glycerylmonostearate, lecithin, acacia gum, xylitol, carboxymethylcellulose, a self-emulsifying agents, glycerol monostearate, glycerol monooleate, Cremophor RH40®, Cremophor EL®, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene). The present disclosure can also exclude one or more of, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides. What can also be excluded is a formulation, composition, device, or method, that comprises pre-gelatinized starch, gelatinized starch, gelatinized corn starch, glycogelatin, alpha-tocopherol, glycogelatin, hemp oil, THC, CBD, gum acacia, sorbitol, xylitol, soy lecithin, a complex of two different gels (one with net negative charge and the other with net positive charge), and compositions that comprise a solvent with a cosolvent.

What can be excluded is pharmaceutical compositions with 1-5% enhancer. What can be excluded is pharmaceutical compositions with 0.5-5% neutralizer, or with any amount of neutralizer. What can be excluded is compositions with greater than 0%-5% by weight isopropyl myristate, or with any amount thereof. What can be excluded is pharmaceutical compositions with 0%-10% by weight carbopol, or with any amount of carbopol. What can be excluded is pharmaceutical compositions with about 10% ethanol, about 15%, about 20%, about 24%, about 30%, about 35%, about 40%, about 45%, about 50%, about 55%, about 60%, about 65%, about 70%, about 75%, about 80%, about 85%, about 90% ethanol. The term “about” can mean, for example, plus or minus one percent, plus or minus two percent, plus or minus four percent, plus or minus six percent, plus or minus eight percent, and so on.

For delivery of cannabinoids, for example, a system of solvent/cosolvent can be ethanol (solvent)/propylene glycol (cosolvent). Solvents can be anhydrous alcohol, ethanol, propanol, or isopropanol. Cosolvent can be propylene glycol or PEG. Ratio of solvent/cosolvent (by weight) can be about 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like. In exclusionary embodiments, the present disclosure can exclude solvent/cosolvent compositions where the ratio is, 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like.

Monolithic Patch Embodiments, as Described in US2017/0071870 of Weimann

The following writing is from US2017/0071870 (Ser. No. 15/265,823) of Weimann, which is incorporated herein by reference in its entirety.

Transdermal monolithic CBD patch formulation: Adhesive polymer: 60-95, CBD: 5-20 Penetration enhancer: 0-20, Adhesive polymer: Acrylate from Henkel, Silicone from Dow Corning, PIB from BASF or Henkel, CBD: pure crystalline powder, Penetration enhancer: Transcutol™ (Diethylene Glycol Monoethyl Ether), Oleic acid, isopropyl palmitate (IPP), dimethylsulfoxide (DMSO), 1,2-propylene glycol (1,2-PG), isopropylmyristate (IPM). In this example, the dry adhesive matrix is 30-50 micrometers thick. The area of the patch can be square or oval. The best size of the patch is about 9.5 cm by about 6 cm.

In a monolithic design, a release liner is coated with a mixture of CBD and a PIB or amine-compatible silicone skin adhesive laminated to the backing material. How the Monolithic CBD Patch Works: Step 1. CBD is dissolved in ethyl alcohol or 1,2 PG and mixed into the adhesive solution and penetration enhancer is added if needed. Step 2. Adhesive mix is dispensed on the release liner by means of “knife-over-roll” coating method and dried in the oven at drying time from 1 min to 3 min or until all residual solvents are below 1 ppm. Step 3. Dried adhesive film is laminated to the backing film by means of nipping and edges are slit for farther die cutting of the patches. Step 4. The laminate is placed on the die cutting machine and proper size patches are cut and later packaged in the pouches and boxes.

How CBD is Delivered from Patch Formulation to the Body Through the Skin

Formulations of monolithic patches were prepared by solubilizing CBD in different adhesives and CBD transdermal flux was performed through human cadaver skin using Franz Diffusion Cell method. We found the highest transdermal flux of CBD from a formulation that comprises PIB adhesive and 10% CBD. This shows that a patch measuring 20 cm² can deliver a daily systemic dose of about 5 mg of CBD.

Exemplary Monolithic Patch Invention Formulations: Formulation 1. 10% CBD in EtOH. Formulation 2. 10% CBD in EtOH/H₂O (9/1). Formulation 3. Penetration enhancers: 1,2 PG, IPP, oleic acid, DMSO.

In a first monolithic-style device, a skin adhesive is mixed with the CBD to define a monolithic mixture of adhesive and CBD. The skin adhesive is coated on a backing that is preferably occlusive. The skin adhesive is preferably a polyisobutylene adhesive, an acrylate adhesive or a mixture of both.

In a second monolithic-style device, a skin adhesive is mixed with the CBD (which may be present as substantially pure CBD or an oil extract of a Cannabis plant which comprises CBD and other cannabinoids) to define a substantially monolithic mixture of adhesive and CBD. The skin adhesive is preferably a polyisobutylene adhesive having a viscosity-average molecular weight ranging from about 30,000 Daltons to about 70,000 Daltons, preferably, from about 35,000 Daltons to about 65,000 Daltons, and more preferably from about 40,000 Daltons to about 60,000 Daltons.

Manufacturing Method for Monolithic Patch

CBD is dissolved in ethyl alcohol or 1,2-propylene glycol (1,2 PG) and mixed into the adhesive solution and penetration enhancer is added if needed. Adhesive mix is dispensed on the release liner by means of “knife-over-roll” coating method and dried in the oven at drying time from 1 min to 3 min or until all residual solvents are below 1 ppm. Dried adhesive film is laminated to the backing film by means of nipping and edges are slit for further die cutting of the patches. The laminate is placed on the die cutting machine and proper size patches are cut and later packaged in the pouches and boxes.

Referring to FIG. 2 of US2017/0071870 (Ser. No. 15/265,823) of Weimann, an example of monolithic-style transdermal drug delivery device 40 for delivering CBD is depicted. Monolithic transdermal device 40 includes a backing 42 of the type described previously with respect to backing 22 of reservoir transdermal device 20. A matrix 44 of skin adhesive mixed with a therapeutically effective amount of CBD is coated on one side of backing 42. The matrix 44 is preferably formulated to adhere the device 20 to the user's skin for a period of no less than about 24 hours while avoiding appreciable skin irritation to the user's skin. A release liner 48 is releasable adhered to matrix 44 on a surface of matrix 44 opposite the surface adhered to backing 42. First side 49 of release liner 48 faces away from matrix 44 and a portion of second side 51 of release liner 48 is adhered to matrix 44. To use the monolithic transdermal device 40, the release liner 48 is peeled away and the exposed surface of adhesive matrix 44 is applied to the skin.

The skin adhesive comprising matrix 44 preferably comprises at least one of an acrylate pressure sensitive adhesive, a polyisobutylene pressure sensitive adhesive, and an amine-compatible silicone pressure sensitive adhesive. Suitable acrylate adhesives include DuroTak 87-2074 and DuroTak 87-2194. Suitable polyisobutylene adhesives include those having a viscosity-average molecular weight ranging from about 30,000 Daltons to about 70,000 Daltons, preferably from about 35,000 Daltons to about 65,000 Daltons, and more preferably from about 40,000 Daltons to about 60,000 Daltons.

Matrix 44 preferably comprises a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 44, more preferably from about two (2) to about six (6) percent by weight of matrix 44, and still more preferably from about three (3) to about four (4) percent by weight of the matrix 44. Mineral oils that are suitable for use as the adhesion/viscosity modifier have a molecular weight ranging from 100 to about 1000 Daltons, more preferably from about 200 to about 600 Daltons, even more preferably from about 350 Daltons to about 450 Daltons, and still more preferably about 400 Daltons. Silicone fluids that are suitable for use as the adhesion/viscosity modifier preferably comprise —OH end-capped polydimethylsiloxanes having a kinematic viscosity at 20 degrees C. ranging from about 100 cSt to about 1000 cSt. Commercially available silicone fluids that may be used as the adhesion/viscosity modifier include the Dow Corning Q7-9120 fluids, which are available in kinematic viscosities (at 20 degrees C.) of 20, 100, 350, 1000, and 12,500 cSt. In preferred examples of silicone adhesion/viscosity modifier, the Q7-9120 100 cSt or 1000 cSt (or mixtures thereof) are used.

Preferred polyisobutylene adhesives are not supplied with mineral oil. In certain preferred examples, the polyisobutylene component of matrix 44 is a Vistanex LM polyisobutylene adhesive. In other preferred examples, the polyisobutylene component of matrix 44 is an Oppanol B13 polyisobutylene adhesive supplied by BASF.

In another example, the adhesive component of matrix 44 may comprise a blend of acrylic adhesive and polyisobutylene adhesive, and preferably, a blend of an acrylic adhesive and a polyisobutylene adhesive having the viscosity-average molecular weight described above (from about 30,000 Daltons to about 70,000 Daltons, preferably from about 35,000 Daltons to about 65,000 Daltons, and more preferably from about 40,000 Daltons to about 60,000 Daltons). When acrylic adhesives are combined with such polyisobutylene adhesives, the amount of acrylic adhesive by weight of the total amount of adhesive in matrix 44 is preferably from about one percent to about 50 percent. In one example, the adhesive component of matrix 44 comprises 80 percent Oppanol B 13 by weight of the total amount of adhesive in matrix 44 and twenty percent Durotak 87-2516 by weight of the total amount of adhesive in matrix 44.

Monolithic device may also include one or more penetration enhancers, including oleic acid, isopropyl palmitate (IPP), DMSO, 1,2 propylene glycol, and isopropyl myristate (IPM). The amount of penetration enhancer preferably ranges from zero to about ten (10) percent by weight of the matrix. In an exclusionary embodiment, the present disclosure can exclude any patch or any formulation that has more than one type of penetration enhancer

The skin contact area of device is preferably at least about 10 cm², more preferably at least about 15 cm², and still more preferably at least about 18 cm². At the same time, the skin contact area of device is preferably no more than about 30 cm², preferably no more than about 25 cm², and still more preferably no more than about 22 cm². At a given flux rate, the skin contact area may be selected to achieve the desired daily dose of CBD (or the dose over whatever time period is of therapeutic interest). The above writing is from US2017/0071870 (Ser. No. 15/265,823) of Weimann.

Knife-Over-Roll Coating

Without implying any limitation on the present invention, knife coating is a process by which a thin liquid coating is formed on a continuous web by the application of an excess of coating liquid which is subsequently metered by a rigid knife held in close proximity to a rigidly supported web. The thickness of the coating depends primarily on the clearance, or gap, between the knife and the web, and upon the geometry of the gap (bevel angle, length). Roll coating is a process by which a thin liquid film is formed on a continuous web by use of two or more rotating rolls, such that the fluid flow in a small gap between a pair of rotating rolls is the primary factor controlling the thickness and uniformity of the coated film. The thickness of the coating depends primarily on the gap between adjacent rolls and their relative speeds. Two basic types of roll coaters are distinguished by the relative direction of roll surface motion in the gap: in forward roll coating the roll surfaces move in the same direction and in reverse roll coating they move in opposite directions. In terms of the flow fields, knife coating is a subset of forward roll coating where one surface is stationary. See, Coyle, D. J (1997) Knife and Roll Coating in Liquid Film Coating (ed. S. F. Kistler and P. M. Schweizer). Chapman & Hall, London; W. Rehnby, M. Gustafsson, M. Skrifvars (June 2008) Conference Paper, Coating of Textile Fabrics with Conductive Polymers for Smart Textile Applications, pages 100-103.

Example of Reservoir Patch Embodiments.

Reservoir Patch Manufacturing Method

The present inventor has used the following manufacturing method for reservoir patch, where the method used the indicated stages:

STAGE 1. Gel dispenser. Dispensing active gel solution on membrane. At this point, the stage of manufacture can be represented by an isolated blob of active gel.

STAGE 2. Heat seal press. Gel is covered with heat sealable film and heat seal around the gel. At this point, the stage of manufacture can be represented by a blob in the center, surrounded by a layer of film.

STAGE 3. Kiss cut press. Kiss cutting along the periphery of the heat seal ring. At this point, the stage of manufacture can be represented by a blob at the center, surrounded by a layer of film and where the interior side of the layer is intact and where the exterior layer is perforated. According to one source, “Kiss cutting is . . . a method for providing a converted adhesive tape solution During the kiss-cutting process, the perimeter of each piece is stamped out by a sharp metal die or by a precision laser beam . . . the cut does not penetrate the piece's backing material (liner) Even though the die or laser makes a clean cut all the way through the usable portion of the material, it merely “kisses” the liner sheet. This allows single or multiple adhesive materials to remain on a liner sheet or roll until the end user is ready to remove them.” (CAN-DO® National Tape, Nashville, Tenn.). According to another source, “Laser kiss cutting is used to cut the top layer of a material without cutting through an attached material. Sticker labels are a good example of laser kiss cutting in action. In this process, the outline of the label can be cut without cutting the release or backing material. Typically, C02 lasers are used for kiss cutting applications. Laser kiss cutting can also be combined with perforating or “through cutting” on a single application. (Preco Kansas, Lenexa, Kans.).

STAGE 4. Kiss cut press. Reservoir is covered with overlay film, and kiss cut along the overlay periphery. At this point in the manufacture, the article of manufacture obtained in STAGE 3 is surrounded by another layer, where this layer is the overlay film.

STAGE 5. Cut through press. Patch is cut through the overextended release liner, for easier peel. At this point, the article of manufacture resembles that obtained in STAGE 4, except that the article of manufacture is chopped into segments, where each segment is suitable for attaching to the skin of a human patient or a human consumer.

Reservoir Patch as Described in US2017/0071870 of Weimann

The following writing, and structure numbers, are from US2017/0071870 (Ser. No. 15/265,823) of Weimann, which is incorporated herein by reference in its entirety. Referring to FIG. 1 of US2017/0071870, a reservoir-style transdermal delivery device 20 for the transdermal delivery of CBD is depicted. Reservoir-style transdermal delivery device 20 comprises a backing 22 and a hydrophilic, porous membrane 24. The backing 22 and hydrophilic, porous membrane 24 are attached to one another so as to define a closed volume which acts as a reservoir 26. A preparation comprising CBD, a liquid carrier, and a gelling agent is disposed in the reservoir 26. First side 34 of the hydrophilic, porous membrane 24 is in contact with the preparation. A second side 36 of the hydrophilic, porous membrane 24 faces away from backing 22 and is coated with a skin adhesive 30. The skin adhesive 30 is preferably formulated to adhere the device 20 to the user's skin for a period of no less than about 24 hours while avoiding appreciable skin irritation to the user's skin. Preferred skin adhesives 30 include amine-compatible, silicone, pressure sensitive adhesives. In certain examples, an amine-compatible silicone skin adhesive 30 is provided which comprises a trimethylsiloxy end-capped reaction product of a silanol end-blocked polydimethylsiloxane and a silicate resin. The skin adhesive is preferably provided as an organic solvent solution comprising from about 50 percent to about 70 percent by weight of solid adhesive in an organic solvent like heptane or ethyl acetate and having a viscosity at 20 degrees C. of from about 400 mPa-s to about 1300 mPa-s, preferably from about 450 mPa-s to about 1250 mPa-s, and more preferably from about 500 mPa-s to about 1200 mPa-s.

A first surface 29 of a release liner 28 is releasably adhered to skin adhesive 30, and a second surface 31 of release liner 28 faces away from skin adhesive 30. Suitable release liners include occlusive polymeric films, such as polyester, polypropylene, coated with a release coating that is releasably adherable to silicone, polyisobutylene, and silicone adhesives. Suitable release coatings on first surface 29 of release liner 28 include fluoropolymers and silicone polymers. Commercially available, coated release liners that are suitable for use as release liner 28 include Scotchpak 1022, 9741, 9744, 9748, and 9755 supplied by 3M of Minneapolis, Minn., and FRA 314 and 315 supplied by Fox River Co. To use the reservoir transdermal device 20, release liner 28 is peeled away from skin adhesive 30, thereby exposing skin adhesive 30, and the device 20 is applied so that the skin adhesive 30 contacts the user's skin.

Suitable examples of amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20 degrees C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 24 that is preferably from about 10 to about 20 g/m², more preferably from about 12-18 g/m², and still more preferably from about 14-16 g/m².

Hydrophilic, porous membrane 24 preferably has a mean flow pore size of no more than about 1 micron, preferably not more than about 0.8 microns, still more preferably no more than about 0.4 microns, and even more preferably no more than about 0.2 microns. At the same time, porous membrane 24 preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233, the entirety of which is hereby incorporated by reference.

In the same or other examples, hydrophilic porous membrane 24 preferably has a porosity of at least about 60 percent, more preferably at least about 65 percent, and still more preferably at least about 70 percent. At the same time, hydrophilic porous membrane 24 preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane 24 preferably has a thickness of no more than about 50 microns, preferably no more than about 40 microns, and even more preferably no more than about 35 microns. At the same time, hydrophilic porous membrane 24 preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, hydrophilic porous membrane 24 preferably has an air permeability as determined by the Gurley Test Method (according to ISO 5636-5) that is preferably at least about 10 sec/50 ml, more preferably at least about 20 sec/50 ml, and still more preferably at least about 25 sec/50 ml. At the same time, hydrophilic porous membrane 24 preferably has an air permeability of no more than about 50 sec/50 ml, more preferably no more than about 40 sec/50 ml, and still more preferably no more than about 35 sec/50 ml.

In the same or other examples, hydrophilic porous membrane 24 preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane 24 preferably has a percent elongation in the machine direction as determined by ASTM D882-12 that is preferably at least about 10 percent, more preferably at least about 15 percent, and still more preferably at least about 20 percent.

Hydrophilic porous membrane 24 preferably comprises at least one polymeric material. In one example, hydrophilic porous membrane 24 comprises a polyolefin polymer and a hydrophilic component that comprises a hydrophilic polymer and optionally, a surfactant. As used herein, the term “hydrophilic” when used to describe a porous membrane refers to a membrane that at 20 degrees C. provides a water flux for demineralized water through the membrane of at least 0.5 liters/(m²hbar).

The content of the polyolefin polymer is preferably less than or equal to 98 percent by weight based on the total dry weight of the membrane 24, and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

In another preferred embodiment, device comprises transdermal patch formulation comprising a reservoir in the shape of a “ravioli” constructed with microporous hydrophilic or hydrophobic membrane on one side and occlusive film on other side.

In embodiments, device comprises transdermal reservoir patch formulation as thixotropic alcohol or alcohol/water solution gelled with hydroxyalkyl cellulose containing CBD at high concentration ranging from 1% to 50% CBD Moreover, device comprises transdermal reservoir patch formulation comprising a reservoir containing thixotropic alcohol or alcohol/water solution gelled with hydroxyalkyl cellulose and containing CBD at a high concentration, ranging from 1% to 50% and skin penetration enhancers in a concentration range of 0% to 10%.

What is also encompassed, is transdermal patch formulation comprising a reservoir in shape of “ravioli” constructed with microporous hydrophilic or hydrophobic membrane on one side and occlusive film on other side where the microporous membrane is coated with thin layer of silicone adhesive. In delivery embodiments, reservoir patch of 20 cm² is capable of systemically delivering CBD at about 0.5 mg/day, about 1.0 mg/day, about 1.5 mg/day, about 2.0 mg/day, about 5.0 mg/day, about 10 mg/day, about 15 mg/day, about 20 mg/day, about 25 mg/day, about 30 mg/day, about 35 mg/day, about 40 mg/day, and the like.

In other delivery embodiments, reservoir patch of 20 square centimeters is capable of systemically delivering CBD at least 0.5 mg/day, at least 1.0 mg/day, at least 1.5 mg/day, at least 2.0 mg/day, at least 5.0 mg/day, at least 10 mg/day, at least 15 mg/day, at least 20 mg/day, about 25 mg/day, about 30 mg/day, at least 35 mg/day, at least 40 mg/day, and so on.

In exclusionary embodiments, the present disclosure can exclude any monolithic patch or reservoir patch that is not capable of delivering CBD (or of any other cannabinoids) to the skin at one of the above-disclosed ranges or above-disclosed values. In exclusionary embodiments, the present disclosure can exclude any monolithic patch or reservoir patch that is shown to be capable of delivering CBD (or of any other cannabinoids) to the skin at one of the above-disclosed ranges or above-disclosed values.

Chemistry of Acrylic Adhesives and Chemistry of Tackifiers of the Present Disclosure

Duro-Tak® 87-2516 is an acrylic copolymer adhesive containing EHA, vinylacetate, and hydroxyethylacrylate. EHA is 2-ethylhexylacrylate (see, U.S. Pat. No. 5,783,208 of Venkateshwaran). Duro-Tak® 87-2516 is an acrylate-vinylacetate copolymer with a hydroxyl group (see, Zhao, Park, Kim, Lee (2002) Drug Devel. Industrial Pharmacol. 28:1125-1131). Duro-Tak® 87-2516 has viscosity of 4350 cp at 41.5% solids (see, US2006/173,124 of Paul). Duro-Tak® 87-2516 is hydroxyfunctional and crosslinked (see, US2002/0058068 of Houze). Duro-Tak® 87-2516 is an acrylate-vinyl acetate self-curing pressure-sensitive adhesive in an organic solvent (see, US2006/0121102 of Chiang).

Duro-Tak 87-4287 is a copolymer with 2-ethylhexyl acrylate as the main repeating monomer unit. Duro-Tak 87-4287 is a copolymer with vinyl acetate and contains OH— functional groups as 2-hydroxyethyl acrylate is also part of the polymer composition (Wolff (2014) Pharm. Res. 31:2186-2202).

Duro-Tak® 87-2287 is a polyacrylate adhesive. According to U.S. Pat. No. 5,693,335 of Xia, “Duro-Tak 87-2287 is a solution polyacrylate adhesive available from National Starch and Chemical Cₒ. Its monomer composition is: vinyl acetate, 28%; 2-ethylhexyl acrylate, 67%; hydroxyethyl acrylate, 4.9% glycidal methacrylate, 0.1%. It contains no crosslinking agent. It is available as a 50% solids solution in ethyl acetate.” See also, U.S. Pat. No. 6,071,531 of Jona. According to U.S. Pat. No. 5,780,050 of Jain, Duro-Tak® 87-2287 is an acrylic adhesive free of acid functional groups. According to US2009/0258061 of Hwang, “Duro-Tak® 87-2287 is an adhesive is derived from a monomer composition of vinyl acetate, 28%; 2-ethylhexyl acrylate, 67%; hydroxyethyl acrylate, 4.9%; and glycidyl methacrylate, 0.1%, see U.S. Pat. No. 5,693,335.”

DuroTak® 87-900A is an acrylic pressure-sensitive adhesive that comprises 2-ethylhexyl acrylate, butyl acetate, t-octyl acrylamide, and methyl methacrylate. This list of chemicals was accepted, as a substitute for “DuroTak® 87-900A” by the patent examiner in file history of US2009/0297590 of Yamagi. According to a Product Selection Guide, DuroTak® 87-900A has no crosslinker, no vinyl acetate, 43% solids, viscosity of 1800 cP (see, DURO-TAK and GELVA Transdermal Pressure Sensitive Adhesives. Product Selection Guide (2013) Henkel Corp., Bridgewater, N.J. (2 pages)). According to Wolff (2014) Pharm. Res. 31:2186-2202, Dura-Tak 87-900A is, “Duro-Tak 87-900A . . . have 2-ethylhexylacrylate as the main repeating monomer unit . . . . Duro-Tak 87-900A contains besides 2-ethylhexylacrylate, butylacrylate, methyl methacrylate and tertiary-octyl acrylamide units.” See also, para. 0031 of Yamagi US2009/0297590. Duro-Tak 87-900A contains 2-ethylhexyl acrylate as the main repeating monomer unit, and also contains butylacrylate, methyl methacrylate and tertiary-octyl acrylamide units (Wolff (2014) Pharm. Res. 31:2186-2202).

Duro-Tak® 87-2510 has been described as, “copolymer: acrylate; functional group: OH; 40.5% solution of noncrosslinking acrylic copolymer, 4500 cps, solubility parameter 16.” (see, Kim, Gwak, Chun (2014) Arch. Pharm. Res. 27:763-768).

Escorez® 5400 is described as, “dicyclopentadiene (DCPD) resin” (see, U.S. Pat. No. 9,296,930 of Hu); “hydrogenated polycyclopentadiene resin” (see, U.S. Pat. No. 9,039,862 of Lotz); a “hydrocarbon tackifying resin, having a molecular weight of about 400 grams/mole, a softening point of 103 degrees C., and a glass transition temperature of about 50 degrees C.” (see, U.S. Pat. No. 9,074,087 of Chen); a “cycloalphiphatic hydrocarbon tackifying resin having a ring and ball softening point from about 100 degrees C. to about 106 degrees C.” (see, U.S. Pat. No. 9,803,113 of Tse).

Escorez® 5400 has the following characteristics: softening point 218.1 degrees F., initial color: 0.6 YI; thermal color stability: 5 hours, 347 degrees F. (175 degrees C.) 6.4 YI, melt viscosity: 320 degrees F. (160 degrees C.) of 800 cP; molecular weight (number average; Mn) 400 g/mol; molecular weight (Mw) 670 g/mol; glass transition temperature (Tg): 126 degrees F. (Product Datasheet, ExxonMobil, Escorez® 5400 Tackifying Resin).

MANUFACTURING. Dermal patches and related medical devices of the present disclosure can be manufactured using one of more of the following machines. The machines used are: An ultra high shear dispersion blade mixer, a hotplate stirrer and magnetic stir bar, a gyroscopic mixer, a custom manufactured lamination machine that uses the solvent casting method to lay down the mixed adhesive formulation to a knife-over-roll coating head. The machine also includes a 5′ oven which, for this formulation, maintains a temperature of 85 degrees C. The web moves at 5′ per minute, resulting in an oven time of exactly 1 minute. The web is then laminated to an occlusive backing and rerolled.

In this context, what is meant is a 5 foot long oven with small (approximately 0.75 inch tall, 12 inch wide) apertures at either end. The coated release liner web moves through the machine (and thus, the oven) at 5 feet per minute, meaning that the coated web is in the oven for 1 minute.

A custom manufactured die-cutting machine which kiss-cuts the patches to a variable shape and size including, but not limited to circles, ovals, squares, rectangles, rounded-edge squares, rounded edge-rectangles, hexagons, rounded-edge hexagons, and other polygons with or without rounded edges of sizes 10 square cm, 15 square cm, 20 square cm, 25 square cm, 30 square cm, 35 square cm, 40 square cm, 45 square cm, 50 square cm, 55 square cm, 60 square cm, 70 square cm, 80 square cm, 90 square cm, 94 square cm, and 100 square cm.

CHEMICALS AND CONCENTRATIONS. Cannabidiol (CBD) concentrations between 1 and 25%, preferably between 5-20%, more preferably between 10-20%, more preferably between 15-20%.

Capsaicin concentrations between 0.01-2%, more preferably between 0.1-1%, more preferably 0.1-0.5%, more preferably 0.1-0.25%.

Menthol concentrations between 0.1-15%, more preferably between 1-10%, more preferably 2.5-5%.

Lidocaine concentrations between 0.1-5%, more preferably between 2.5-5%, more preferably 4% in an over-the-counter (OTC) product and 5% in a prescription-only product.

Diclofenac concentrations between 0.1-2%, more preferably 0.75-1.5%, more preferably between 1-1.25%.

Camphor concentrations between 0.1-15%, more preferably between 1-10%, more preferably between 2.5-5%.

Methyl Salicylate concentrations between 0.1-15%, more preferably between 2.5-15%, more preferably between 5-10%.

Ascorbyl palmitate is an antioxidant in formulations of the present disclosure. Ascorbyl palmitate can prevent decarboxylation of cannabidiolic acid (CBDA), where the decarboxylation product is cannabidiol (CBD). Compositions, formulations, dermal patches, medical devices, and related methods of the present disclosure can comprise, e.g., ascorbyl palmitate (or, alternatively, can comprise butylated hydroxytoluene (BHT), alpha-tocopherol, tocopheryl acetate, or any other antioxidant) at a concentration of: zero %, 0.005% (wt./wt.), 0.01% (wt./wt.), 0.02% (wt./wt.), 0.025% (wt./wt.), 0.03% (wt./wt.), 0.04% (wt./wt.), 0.05% (wt./wt.), 0.06% (wt./wt.), 0.07% (wt./wt.), 0.08% (wt./wt.), 0.09% (wt./wt.), 0.1% (wt./wt.), 0.15% (wt./wt.), 0.20% (wt./wt.), or at a concentration of about 0.005% (wt./wt.), about 0.01% (wt./wt.), about 0.02% (wt./wt.), about 0.025% (wt./wt.), about 0.03% (wt./wt.), about 0.04% (wt./wt.), about 0.05% (wt./wt.), about 0.06% (wt./wt.), about 0.07% (wt./wt.), about 0.08% (wt./wt.), about 0.09% (wt./wt.), about 0.1% (wt./wt.), about 0.15% (wt./wt.), about 0.20% (wt./wt.), and so on. In this context, the word “about” can optionally mean the range of concentrations that encompasses a given value, and where this range is between the immediate lower value in the above list (the list that does not contain the word “about”) and the immediate greater value in the above list (the list that does not contain the word “about”).

The present disclosure also provides dermal patches that do not contain any composition or formulation, and also provides medical devices that do not contain any composition or formulation. These embodiments take the form of the inventive dermal patch, where the composition or formulation has not yet been added.

In exclusionary embodiments, compositions, formulations, dermal patches, medical devices, and related methods of the present disclosure can exclude any composition, formulation, dermal patches, medical devices, that contains ascorbyl palmitate at any one of the above concentrations, or that contains ascorbyl palmitate at a level below any one of the above-disclosed concentrations, or that contains ascorbyl palmitate at a level above any one of the above-disclosed concentrations.

Chemical mechanisms of the antioxidant action of butylated hydroxytoluene (BHT), alpha-tocopherol, and tocopheryl acetate are disclosed (see, pages 619 to 634 of Brody (1999) Nutritional Biochemistry, 2nd ed., Academic Press, San Diego). Concentrations of these other antioxidants, in terms of inclusionary embodiments and also in terms of exclusionary embodiments can be defined by values defined in the above paragraph relating to ascorbyl palmitate.

MATRIX EMBODIMENTS. In some embodiments, dermal patch and medical devices of the present disclosure is a “drug-in-adhesive patch” and does not include any matrix. In exclusionary embodiments, compositions, dermal patches, medical devices, and related methods of the present disclosure do not include any matrix. Alternatively, compositions, dermal patches, medical devices, and related methods of the present disclosure includes a hydrogel matrix, or alternatively some other kind of matrix.

EXCIPIENTS, SOLVENTS, AND ENHANCERS. In one class of embodiments, the only excipient is the up to 6% Transcutol (diethylene glycol monoethyl ether), which is used here as a permeation enhancer. The solvent used for the active ingredient (and Transcutol) dissolution is Ethanol, for the adhesive, Heptane, and as a cosolvent, Ethyl Acetate. These solvents are removed via evaporation in an oven, and residual solvent concentrations for each should be no higher than 50 micrograms per gram.

In some embodiments, the only permeation enhancer is Transcutol at up to 6% w/w. Our adhesive mixture makes up 79.9% of the final patch weight (excluding the backing and the release liner). Of that 79.9%, 92.5% is Henkel DuroTak 87-6908 polyisobutylene adhesive and 7.5% is Henkel DuroTak 87-2074 acrylate adhesive. In exclusionary embodiments, compositions, dermal patches, medical devices, and related methods of the present disclosure can exclude any composition.

### Examples of the Present Invention

Preferred formulations for use in Applicant's topical patch products. The patch product is a 4% lidocaine, 4% menthol topical patch product that is available over-the-counter (OTC). The product is a drug-in-adhesive matrix-type patch that delivers lidocaine and menthol topically using a pressure-sensitive drug delivery adhesive with known stabilizers and enhancers. Each of the applicant's adhesive patch products is a 50 cm² form factor, intended for patient use over a 24-hour cycle. Terocin is a 4% lidocaine, 4% menthol topical patch product that is available by prescription. Terocin is a hydrogel-type patch that does not contain any known anti-inflammatory compounds. Each Terocin patch is intended for patient use in comparatively shorter cycles of 8 hours (to minimize skin irritation associated with high loading of lidocaine at 600 mg and menthol at 600 mg per patch). Terocin has a larger form factor of 96 cm² with adhesive strips at the perimeter length of each patch product.

Formulations (Initial Embodiment). This describes an embodiment of Applicant's drug-in-adhesive transdermal patch. The adhesive is a mixture of Polyisobutylene adhesive and acrylate adhesive in heptane and/or ethyl acetate (Henkel DuroTak 87-6908 and Henkel DuroTak 87-2074, respectively) and comprising up to 79.9% of the solid patch adhesive matrix. Cannabidiol in isolate form of purity greater than 99.5% comprised up to 15% of the adhesive matrix, a permeation enhancer comprised up to 6% of the adhesive matrix, and lidocaine and menthol comprised 4% each of the adhesive matrix. Ascorbyl palmitate comprises 0.1% of the adhesive matrix. Heptane is used as a solvent for the adhesive, ethanol is used as a solvent for the active ingredients and enhancers, and ethyl acetate is used as a cosolvent. The solvent casting method is used, and the adhesive mixture is cast via knife-over-roll onto a siliconized PET release liner (Fox River FRA-316) at a thickness of about 0.007 inches. The solvents in the coated liner are evaporated by the use of an oven kept at 85° C. for no less than 1 minute. Residual solvents were well below acceptable maximums after this point and the total coat weight of the mixture was measured at approximately 50 grams per square meter (gsm). At this point, the release liner is laminated to an occlusive backing of polyethylene foam (UFP Technologies). After 24 hours, the laminated roll is die-cut to 50 square centimeters, rounded-edge rectangles, and finally packaged.

Formulations (Second Generation Embodiment). Second generation embodiment is similar to the above-described initial embodiment, except that the adhesive used was changed from only Dupont Liveo BIO-PSA AC7-4301 to a mixture of Dupont Liveo BIO-PSA AC7-4301 and Dupont Liveo BIO-PSA AC7-4201, at a ratio of 85% to 15%. This change alleviates the problem of cold flow.

Results from tests on the Applicant's patch product and on the TEROCIN patch product embodiment, using a Franz Diffusion Cell. In vitro delivery of the topical anesthetic lidocaine was tested using the Franz Diffusion Cell method with dermatomed cadaver skin as a membrane for both the applicant's patch product and Terocin patch product. Results demonstrated a higher in vitro flux per unit area for applicant's patch when compared to Terocin patch over each product's recommended use period. The applicant's patch flux measured at an average of 72.6 plus or minus 15.2 micrograms per square centimeter, and Terocin patch flux measured at an average of 39.7 plus or minus 0.8 micrograms per square centimeter. For full results see Table 1. Over the total patch area, this would result in a cumulative flux of 3.63 plus or minus 0.76 mg for applicant's and 3.8 plus or minus 0.08 mg for Terocin patch. The results fall within statistical equivalence. The reason why the applicant's patch and Terocin patch products deliver statistically equivalent amounts of lidocaine in vitro despite applicant's higher in vitro flux per unit area is due to the 92% larger surface area of the Terocin patch. Test data results are disclosed in the following paragraph.

Data on lidocaine flux from Applicant's patch and data on lidocaine flux from Terocin patch. Three samples of the applicant's patch were tested and three samples of the Terocin patch were tested as described in the above paragraph. Tests used the Franz Diffusion Cell method described in Bartosova and Bajgar (2012) Transdermal drug delivery in vitro using diffusion cells. Current Medicinal Chemistry. 19:4671-4677. Lidocaine Flux (micrograms per square centimeter) for three different samples of Applicant's patch was, 77.5 ug/cm², 55.5 ug/cm², and 84.7 ug/cm². Lidocaine Flux (micrograms per square centimeter) for three different samples of Terocin patch was, 40.6 ug/cm², 39.1 ug/cm², and 39.3 ug/cm².

Discussion of the lidocaine flux test results that were acquired from tests on the Applicant's patch and from the Terocin patch. Over their recommended use periods, the applicant's and Terocin patch products deliver a statistically equivalent amount of lidocaine, as determined by the Franz Diffusion Cell analytical technique. The applicant's patch achieves statistically equivalent lidocaine delivery with a dose of 9 mg of lidocaine per patch (40% efficiency), compared against Terocin with a dose per patch of 600 mg (0.6% efficiency). Both patch products rely on menthol at 4% as a topical analgesic, a sensory agent, and permeation enhancer for lidocaine flux.

With a smaller form factor (applicant's patch at 50 centimeters squared and Terocin patch at 96 centimeters squared), and much less likelihood of adverse skin reactions from over-exposure to lidocaine or menthol (applicant's patch at 9 mg lidocaine versus Terocin patch at 600 mg lidocaine), patients may prefer applicant's patch for convenience of application of the patch in the affected area, as recommended by the prescribing physician. Patients may also be susceptible to lower incidence of the undesirable side effects of skin irritation from a drug load that remains in contact with the skin but does not permeate the dermal layer. Lidocaine doses are less effective in the presence of inflammation. In order to enhance the efficacy of lidocaine delivered topically, applicant's patch products contain cannabidiol (CBD) at a dose rate of 40 milligrams per patch. Terocin patch products do not contain any known anti-inflammatory compounds.

MONOLITHIC-STYLE PATCH; RESERVOIR-STYLE PATCH. In embodiments, the dermal patch of the present disclosure is a monolithic-style patch. In less preferred embodiments, the dermal patch of the present disclosure is a reservoir-style patch. In exclusionary embodiments, compositions, dermal patches, medical devices, and related methods of the present disclosure can exclude reservoir-style patches and can exclude compositions intended to be used in a reservoir patch (intended for delivery to the skin by the reservoir patch).

PHARMACOLOGICAL EFFECTS. Cannabidiol (CBD) acts to reduce inflammation (rubor, tumor, calor) as well as chronic pain (dolor). The latter is the main subject of study for the Remy patch product and its patient evaluations. Capsaicin falls under the same general principle, although no patches have been presented to patients. The anti-inflammatory effects of Capsaicin are comparative in effect to those of the prescription NSAID Diclofenac. In studies, capsaicin has been shown to significantly reduce pain and inflammation due to arthritis, including a 57% reduction in pain severity for Rheumatoid Arthritis patients. The likely method of anti-inflammatory action of capsaicin lies in its reduction of Substance P (SP) levels in the body. Local SP production results in neurogenic inflammation. The reduction of both systemic and local SP levels by capsaicin likely results in a reduction in the inflammatory response.

The present invention is not to be limited by compositions, reagents, methods, diagnostics, laboratory data, and the like, of the present disclosure. Also, the present invention may not be limited by any preferred embodiments that are disclosed herein.

What is claimed:

1. A dermal patch that is capable of adhering to the skin of a human user, and also capable of delivering pharmaceutically effective amounts of each of lidocaine, capsaicin, menthol, and at least one cannabinoid to said skin, wherein said dermal patch comprises:
(i) A composition that comprises lidocaine, capsaicin, menthol, and at least one kind of cannabinoid,
(ii) A backing comprising a first surface that faces said composition and a second surface that faces the atmosphere,
(iii) A skin adhesive,
(iv) A release liner having a first surface that faces said composition and a second surface that faces the atmosphere, wherein the release liner is capable of being peeled away thereby exposing the patch's adhesive, wherein said exposed adhesive is capable of adhering to the user's skin.

2. The dermal patch of claim 1, wherein said at least one kind of cannabinoid comprises cannabidiol (CBD).

3. The dermal patch of claim 1 that further comprises hemp oil, and where the hemp oil is mixed with said composition that comprises lidocaine, capsaicin, menthol, and at least one kind of cannabinoid.

4. The dermal patch of claim 1, wherein said composition further comprises one or more penetration enhancers, and wherein said one or more penetration enhancers is selected from isopropyl palmitate (IPP), DMSO, 1,2 propylene glycol, isopropyl myristate (IPM), and diethylene glycol monoethylether, dihydromyricetin, diethylene glycol monoethyl ether (Transcutol®), triacetin, dipropylene glycol, isophytol, phytol, oleic acid, a terpene, ethanol, azone (azone is 1-dodecyl azepan-2-one), oleic acid, dimethylsulfoxide (DMSO), and limonene.

5. The dermal patch of claim 1, wherein said skin adhesive takes the form of a mixture of skin adhesive and one or more penetration enhancers selected from isopropyl palmitate (IPP), DMSO, 1,2 propylene glycol, isopropyl myristate (IPM), and diethylene glycol monoethylether, dihydromyricetin, diethylene glycol monoethyl ether (Transcutol®), triacetin, dipropylene glycol, isophytol, phytol, oleic acid, a terpene, ethanol, azone (azone is 1-dodecyl azepan-2-one), oleic acid, dimethylsulfoxide (DMSO), and limonene.

6. The dermal patch of claim 1, wherein the skin adhesive comprises at least one of:
(i) An acrylate pressure sensitive adhesive,
(ii) A Polyisobutylene (PIB) pressure sensitive adhesive,
(iii) An amine-compatible silicone pressure sensitive adhesive, and
(iv) An amine-compatible silicone skin adhesive comprising a trimethylsiloxy end-capped reaction product of a silanol end-blocked polydimethylsiloxane and a silicate resin.

7. The dermal patch of claim 1, wherein said skin adhesive is provided as an organic solvent solution comprising from about 20 percent to about 70 percent by weight of solid adhesive in an organic solvent like heptane or ethyl acetate and having a viscosity at 20 degrees C. of from about 400 mPa-s (millipascal-seconds) to about 8000 mPa-s, from about 850 mPa-s to about 7000 mPa-s, or from about 1250 mPa-s to about 6500 mPa-s.

8. The dermal patch of claim 1 that is a monolithic-style dermal patch.

9. The dermal patch of claim 1 that is a monolithic-style dermal patch comprising a matrix formulated to maintain adhesion of the dermal patch to the user's skin for a period of at least 24 hours, wherein the release liner of said monolithic-style dermal patch is releasably adhered to the matrix.

10. The dermal patch of claim 1 that is a reservoir-style dermal patch.

11. The dermal patch of claim 1 that is a reservoir-style dermal patch that comprises a hydrophilic porous membrane,
wherein the backing and the hydrophilic porous membrane are attached to one another to define a closed volume that acts as a reservoir, wherein said composition is disposed in the reservoir,
wherein said hydrophilic porous membrane has a first side that contacts said reservoir, and wherein the hydrophilic porous membrane has a second side that faces away from the backing and is coated with skin adhesive.

12. The dermal patch of claim 1, wherein the release liner comprises an occlusive polymeric film, such as polyester, polypropylene.

13. The dermal patch of claim 1, wherein the release liner is coated with a release coating that is releasably adherable to silicone, polyisobutylene, and silicone adhesives.

14. The dermal patch of claim 10, comprising 4% lidocaine, 4% menthol

15. The dermal patch of claim 14, demonstrating higher Lidocaine flux between at least about 20% and 40% over Terocin® brands of products using the Franz Diffusion cell method, using dermatomal cadaver skin as a membrane.

16. The dermal patch of claim 11, comprising 4% lidocaine, 4% menthol

17. The dermal patch of claim 16, demonstrating higher Lidocaine flux between at least about 20% and 40% over Terocin® brands of products using the Franz Diffusion cell method, using dermatomal cadaver skin as a membrane.

18. A method of treatment, comprising the steps of applying a patch to the skin of a subject; having menthol 4% and lidocaine 4% along with CBD; and allowing said patch to remain for at least about 23-27 hours on the skin.
